(19) United States
(12) Patent Application Publication
(10) Pub. No.: US 2003/0166161 A1
(43) Pub. Date: Sep. 4, 2003

(54) Gene that imparts selective proliferative activity

(76) Inventors: Keiya Ozawa, Tochigi (JP); Katsuhisa Itoh, Ibaraki (JP); Tsuneaki Sakata, Osaka (JP); Yasuji Ueda, Ibaraki (JP); Mamoru Hasegawa, Ibaraki (JP); Takeyuki Nagashima, Ibaraki (JP); Akihiro Kume, Tochigi (JP)

Correspondence Address: CLARK & ELBING LLP, 101 FEDERAL STREET, BOSTON, MA, 02110, US

(21) Appl. No.: 10/100,471

(22) Filed: Mar. 18, 2002

Related U.S. Application Data
(63) Continuation-in-part of application No. 09/577,084, filed on May 24, 2000.
Application No. 09/577,084 is a continuation-in-part of application No. 09/142,305, filed on Sep. 10, 1999.

(30) Foreign Application Priority Data
Mar. 5, 1997 (WO) PCT/JP97/00687

(51) Int. Cl. A61K 38/17; C07H 21/04; C12P 21/04; C12N 5/06; C07K 14/72

(52) U.S. Cl. 435/69.7; 435/320.1; 435/325; 514/12; 530/350; 536/23.5

(57) ABSTRACT

Selective amplification of cells is enabled by introducing into cells a gene encoding a fusion protein comprising (a) a ligand-binding domain, (b) a domain that associates when the ligand binds to the domain of (a), and (c) a domain that imparts proliferation activity to the cells upon the association and stimulating the cells with the ligand.

### TECHNICAL FIELD

The present invention relates to the field of genetic engineering, particularly the field of gene therapy.

### BACKGROUND ART

Various methods have so far been devised to treat diseases caused by congenital or acquired genetic defects, namely, gene disorders. In gene therapy, one such method, a defective gene itself is substituted by or supplemented with a normal gene in order to fundamentally cure gene disorders. It is important for the success of gene therapy to introduce a normal gene accurately into target cells and to express the introduced gene accurately. The conventionally used vectors for introducing a normal gene into target cells are viral vectors such as retrovirus vectors, adenovirus vectors, and adeno-associated virus vectors, and non-viral vectors such as liposomes. However, all have some shortcomings such as low gene introduction efficiency into target cells. Furthermore, they are often inadequate for treatment because of additional disadvantages such as poor expression efficiency of an introduced gene. In adenosine deaminase (ADA) deficiency, the normal ADA gene-introduced cells are expected to acquire a survival advantage or a growth advantage and gradually become dominant as a result of in vivo selection. In such a case, it may be possible to obtain gradual treatment effects despite the poor gene introduction efficiency. However, it is often necessary to introduce a gene for treatment that cannot be selected in vivo. It has thus been desired to establish a system that enables selective amplification of cells containing an introduced gene.

Although G-CSF was traditionally considered as a cytokine (a hematopoietic factor) that selectively proliferates neutrophils, it has recently been reported that the administration of G-CSF increases not only neutrophils but also the hematopoietic stem cell/precursor cell pool in the body (Rinsho Ketsueki (Clinical Blood), 35, 1080 (1994)). The mechanism of manifestation of the G-CSF function has been reported to be dimerization of a G-CSF receptor that takes place upon activation of the G-CSF receptor by stimulation with G-CSF (Proc. Growth Factor Res., 3 (2), 131-141 (1991)). It has also been reported that the G-CSF receptor has a proliferation-inducing domain and a differentiation-inducing domain (Cell, 74, 1079-1087 (1993)). Moreover, like the G-CSF receptor, an estrogen receptor is known to be activated through dimerization (J Biol. Chem., 264, 2397-2400 (1989)), and there is a report that expression of a fusion protein between the estrogen receptor and c-Abl tyrosine kinase in the cell resulted in activation of the c-Abl tyrosine kinase (The EMBO Journal, 12, 2809-2819 (1993)).

### DISCLOSURE OF THE INVENTION

The present invention seeks to overcome the problem of poor gene introduction efficiency by selectively amplifying in vivo or ex vivo hematopoietic stem cells into which a gene for treatment has been introduced. The objective of the invention is to provide a fundamental technique for gene therapy targeting hematopoietic stem cells.

In the field of gene therapy today, there are numerous problems to be overcome concerning the efficiency of gene introduction into target cells and the expression efficiency of the introduced gene. It is therefore obvious that establishing a system for selectively amplifying only the target cells containing the introduced gene will produce a major breakthrough. In particular, if such a system is established for hematopoietic stem cells, which are the origin of many blood cells such as red blood cells or white blood cells and which are considered to be the most preferable target cells for gene therapy, it would contribute significantly to the field of gene therapy.

G-CSF, which was traditionally thought to be a cytokine (a hematopoietic factor) that selectively proliferates neutrophils, can also proliferate hematopoietic stem cells. The G-CSF receptor dimerizes itself when it is activated. Considering these facts, the present inventors have thought of a system for amplifying hematopoietic stem cells through dimerization of a genetically engineered G-CSF receptor. Also based on the fact that the estrogen receptor dimerizes itself upon stimulation with estrogen, the present inventors have thought of constructing a chimeric gene between the G-CSF receptor gene and the estrogen receptor gene, introducing the chimeric gene into cells, and externally stimulating the cells by estrogen to forcibly dimerize the G-CSF receptor portion of the chimeric gene product.

Thus, the present invention was completed by developing a new system for selectively amplifying hematopoietic stem cells into which a gene has been introduced by activating the G-CSF receptor portion of the chimeric gene product through external stimulation with estrogen.

Furthermore, the present inventors have constructed a mutant receptor (TmR) which specifically binds to 4-hydroxytamoxifen (Tm), and have replaced the above-mentioned estrogen receptor with the TmR, to overcome influences of the endogenous estrogen on this system. Thus, the present inventors have developed the system in which gene-modified hematopoietic stem cells selectively expanded as a result that G-CSF receptor of the chimeric fusion protein is activated by stimulation of exogenous Tm without the influences of the endogenous estrogen.

The present invention relates to a fusion protein comprising a ligand-binding domain, a domain that associates when a ligand binds to the ligand-binding domain, and a domain that imparts proliferation activity to a cell upon the association; a vector comprising a gene encoding the fusion protein; a cell containing the vector; and a method for selectively proliferating the cell either in vivo or ex vivo by exposing the cell to a steroid hormone. Furthermore, when the vector contains an exogenous gene, the present invention relates to a method for selectively proliferating a cell into which the exogenous gene has been introduced.

More specifically, the present invention relates to:

(1) A fusion protein comprising (a) a ligand-binding domain, (b) a domain that associates when a ligand binds to the domain of (a), and (c) a domain comprising a cytokine receptor or a part thereof that imparts proliferation activity to a cell upon the association.

(2) The fusion protein of (1), wherein the “domain comprising a cytokine receptor or a part thereof that imparts proliferation activity to a cell upon the association” is derived from a G-CSF receptor or a c-mpl.

(3) The fusion protein of (1), wherein the “ligand-bindingdomain” is derived from a steroid hormone receptor.

(4) The fusion protein of (3), wherein the steroid hormone receptor is an estrogen receptor.

(5) The fusion protein of (1), wherein the “ligand” is a tamoxifen, the derivative thereof, or the metabolite thereof and the “ligand-binding domain” and “a domain that associates when a ligand binds to said domain” are derived from a mutant estrogen receptor that is unresponsive to a estrogen and that is responsive to a tamoxifen, the derivative thereof, or the metabolite thereof.

(6) A DNA encoding the fusion protein of (1).

(7) A vector comprising a DNA of (6).

(8) A cell carrying the vector of (7).

(9) A method for selectively proliferating the cell of (8), which comprises exposing the cell of (8) to a ligand capable of acting on the “ligand-binding domain” of the fusion protein of (1).

(10) A vector comprising a desired exogenous gene and a gene encoding a fusion protein comprising (a) a ligand-binding domain, (b) a domain that associates when a ligand binds to the domain of (a), and (c) a domain that imparts proliferation activity to a cell upon the association.

(11) The vector of (10), wherein the “domain that imparts proliferation activity to a cell upon the association” is derived from a cytokine receptor.

(12) The vector of (11), wherein the cytokine receptor is a G-CSF receptor or a c-mpl.

(13) The vector of (10), wherein the “ligand-binding domain” is derived from a steroid hormone receptor.

(14) The vector of (13), wherein the steroid hormone receptor is an estrogen receptor.

(15) The vector of (10), wherein the “ligand” is a tamoxifen, the derivative thereof, or the metabolites thereof and the “ligand-binding domain” and “a domain that associates when a ligand binds to said domain” are derived from a mutant estrogen receptor that is unresponsive to a estrogen and that is responsive to a tamoxifen, the derivative thereof, or the metabolite thereof.

(16) The vector of (10), wherein the “gene encoding a fusion protein” and the “exogenous gene” are located on the same molecule.

(17) The vector of (10), wherein the “gene encoding a fusion protein” and the “exogenous gene” are located on separate molecules.

(18) A cell carrying the vector of (10).

(19) A method for selectively proliferating the cell of (18), which comprises exposing the cell of (18) to a ligand capable of acting on the “ligand-binding domain” of the fusion protein encoded by the gene contained in the vector of (10).

(20) A kit comprising (a) the vector of (7) or (10), and (b) a ligand capable of acting on the “ligand-binding domain” of the fusion protein encoded by the gene contained in the vector.

Any ligand can be used in the present invention as long as it acts on a specific protein to cause association of the protein, but a steroid hormone is preferable. Examples of the steroid hormone include estrogens, TPO, androgens, progesterone, glucocorticoids, and mineral corticoids. They are used in combination with their respective receptor proteins. It is also possible to use a synthetic compound as a ligand. For example, a tamoxifen, the derivative thereof (ex. Tremifen), or the metabolite thereof (ex. 4-hydroxytamoxifen) can be preferably used in combination with a mutant estrogen receptor lacking estrogen responsiveness. The mutant estrogen receptor having substitution of glycine-525 with other amino acid, for example arginine, lysine, can be preferably used [Mol. Endocrinol.,(1993) 7: 232-40].

Any cytokine receptor can also be used in the present invention as long as it imparts proliferation activity to a cell upon association. Examples of the cytokine receptor are those belonging to the cytokine receptor family including G-CSF and c-mpl and those belonging to the tyrosine kinase receptor family including c-kit and flk2/flt3.

As the “domain which imparts proliferation activity to a cell” of the fusion protein according to the present invention, it is possible to use a molecule that transmits the intracellular proliferation signal, for example, an entire molecule of a cytokine receptor. It is also possible to use only a domain in the molecule that imparts proliferating activity to a cell. The latter approach is advantageous in proliferating the cell as it is because the domain proliferates the cell into which the fusion protein-coding DNA has been introduced without differentiating it. Furthermore, the vector used in the present invention includes not only a single vector molecule containing the fusion protein-coding DNA and a single vector molecule containing the fusion protein-coding DNA and the exogenous gene, but also includes a vector system of multiple vector molecules comprising a combination of a vector containing the fusion protein-coding DNA and a vector containing the exogenous gene, for example, a binary vector system. Such a vector system of multiple vector molecules is usually introduced into a cell by co-transformation.

When a DNA encoding the fusion protein and an exogenous gene are inserted into the same vector, they may be made into a dicistronic form containing an internal ribosome entry site (IRES) (published PCT Application in Japan No. Hei 6-509713). For example, it is possible to use a vector having a structure containing, from 5′ to 3′, a promoter, an exogenous gene, IRES, and a DNA encoding the fusion protein or a vector having a structure containing, from 5′ to 3′, a promoter, a DNA encoding the fusion protein, IRES, and an exogenous gene. The former type is generally used to allow most of the cells expressing the fusion protein gene to express the exogenous gene.

Moreover, in the present invention, the cell into which the vector is introduced includes hematopoietic stem cells, lymphocyte, and cells other than these blood cells. In particular, hematopoietic stem cells that can self-proliferate are preferable in the present invention. Although the exogenous gene to be introduced into the cell in the present invention is not particularly limited, a normal gene corresponding to a defective gene is generally used in the field of gene therapy.

### BRIEF DESCRIPTION OF THE DRAWINGS

FIG. 1(A) shows a chimeric molecule between the G-CSF receptor and the estrogen receptor (GCRER). (B) shows a mutant of the chimeric molecule between the G-CSF receptor and the estrogen receptor, deficient in the 5th through the 195th amino acids of the G-CSF receptor (GCRΔ(5-195)/ER). (C) shows a mutant of the chimeric molecule between the G-CSF receptor and the estrogen receptor, deficient in the 5th through 195th amino acids and the 725th through 756th amino acids of the G-CSF receptor (GCRΔ(5-195, 725-756)/ER).

FIG. 2 shows a retrovirus vector “pMX” in which a chimeric gene between the G-CSF receptor and the estrogen receptor has been incorporated.

FIG. 3 shows proliferation of the Ba/F3 cells transformed with “pCMX-GCRER” with the passage of time.

FIG. 4 shows proliferation of the Ba/F3 cells transformed with “pCMX-GCRER” with the passage of time; the cells were stimulated with various concentrations of estradiol.

FIG. 5 shows proliferation of the Ba/F3 cells transformed with “pCMX-GCRΔ(5-195)/ER” with the passage of time.

FIG. 6 shows plasmid “pCMX-GCRER-IRES-CD24.”

FIG. 7 shows plasmid “pCMX-GCRΔ(5-195)/ER-IRES-CD24.”

FIG. 7 shows plasmid “pCMX-GCRΔ(5-195, 725-756)/ER-IRES-CD24.”

FIG. 9 shows the expression of CD24 in the Ba/F3 cells into which “pCMX-GCRA(5-195)/ER-IRES-CD24” has been introduced, detected by flow cytometry. The upper panel shows the results from the Ba/F3 cells into which “pCMX-GCRΔ(5-195)/ER-IRES-CD24” has been introduced; the lower panel shows the result from the Ba/F3 cells into which “pCMX-GCRΔ(5-195)/ER” has been introduced as a control. (Note that the data also contain the signal from propidium iodide that was used to detect dead cells.)

FIG. 10 is a microscopic photograph showing granulocyte-macrophage lineage colonies derived from bone marrow cells into which “vMXGCRER” has been introduced.

FIG. 11 is a microscopic photograph showing erythroblastic colonies derived from the bone marrow cells into which “vMXGCRΔ(5-195)/ER” has been introduced.

FIG. 12 is a microscopic photograph showing the Wright-Giemsa-stained macrophage which have differentiated from the bone marrow cells into which “vMXGCRER” was introduced.

FIG. 13 is a microscopic photograph showing the Wright-Giemsa-stained erythroblasts which have differentiated from the bone marrow cells into which “vMXGCRΔ(5-195)/ER” was introduced.

FIG. 14 is structures of the molecules involved in this study. (A) the murine G-CSF receptor (GCR), (B) a GCR derivative deleting the G-CSF-binding site (ΔGCR), (C) a mutant estrogen receptor specific for 4-hydroxytamoxifen (TmR), and (D, E) the fusion proteins between GCR and TmR (GCRTmR) or ΔGCR and TmR (ΔGCRTmR). Extracellular, extracellular region; G, G-CSF-binding site (amino acids 5-195); TM, transmembrane domain; Cytoplasmic, cytoplasmic portion; TA, transactivation domain; DNA, DNA-binding domain; HBD, hormone-binding domain; G525R, arginine substitution for glycine-525 in mouse estrogen receptor.

FIG. 15 shows retroviral vectors used in this study. (A) MSCV/GCRTmR-IRES-CD8a. (B) MSCV/ΔGCRTmR-IRES-CD8a. (C) MSCV/GCRTmR-IRES-EGFP. (D) MSCV/IRES-EGFP. LTR, long terminal repeat; GCRTmR, cDNA for GCRTmR; ΔGCRTmR, cDNA for 66 GCRTmR; IRES, encephalomyocarditis virus-derived internal ribosome entry site; CD8a, cDNA for murine CD8a.

FIG. 16 shows Western blot analysis of BaF/GCRTmR and BaF/ΔGCRTmR cells. Lysates of control Ba/F3 (C; lane 1), BaF/GCRTmR clones (lanes 2-4) and BaF/ΔGCRTmR clones (lanes 5-7) were electrophoresed and transferred onto PVDF membranes. The membranes were hybridized with an anti-GCR (top) or an anti-ER (bottom) antibody, and GCRTmR fusion protein (ca. 140 kDa) and ΔGCRTmR fusion protein (ca.120 kDa) were visualized with an ECL kit (Amersham).

FIG. 17 shows growth curves of parental Ba/F3 cells (A), BaF/GCRTmR clone 1 (B), clone 2 (C), clone 3 (D), BaF/ΔGCRTmR clone 1 (E), clone 2 (F) and clone 3 (G). Cells were incubated with IL-3 (closed squares), G-CSF (closed circles), Tm (closed triangles), E₂ (closed diamonds) or no stimulator (open squares). The graphs represent cumulative A₄₉₀-A₆₂₀ values of XTT assay (means±SD of triplicate determinants).

FIG. 18 shows growth curves of long-term culture of GCRTmR-transduced Ba/F3 cells. BaF/GCRTmR clone 1 (squares), clone 2 (circles) and clone 3 (triangles) were incubated with Tm (closed symbols) for 36 days. Aliquots of the cells were periodically deprived of Tm and evaluated for viability (open symbols). The growth curves represent cumulative A490-A620 values of XTT assay (means±SD of triplicate determinants).

FIG. 19 shows dose-dependence assay of GCRTmR-transduced Ba/F3 cells. BaF/GCRTmR clone 1 (squares), clone 2 (circles) and clone 3 (triangles) were incubated with various concentrations of E2 (A) or Tm (B). XTT assay was performed on day zero and day four, and graphs represent the ratios of day four A₄₉₀-A₆₂₀ to day zero A₄₉₀-A₆₂₀ (means±SD of triplicate determinants).

FIG. 20 shows structures of the vectors used in this study. These vectors express the fusion proteins between (A) ΔGCR and ER, (B) Mpl and ER, (C) ΔGCR-Mpl and ER, (D) ΔGCR-Mpl and TmR.

FIG. 21 shows cell proliferation assay of Ba/F3 cells transduced with the chimeric genes, ΔGCR-ER(left), Mpl-ER(right)

FIG. 22 shows cell proliferation assay of Ba/F3 cells transduced ΔGCR-Mpl-ER.

FIG. 23 shows proliferation of Ba/F3 cells expressing ΔGCR-Mpl-TmR. (Left)Growth curves of parental Ba/F3 cells expressing ΔGCR-Mpl-TmR. (Right)Long-term culture of Ba/F3 cells transduced with ΔGCR-Mpl-TmR. A representative clone of BaF/ΔGCRmplTmR was cultured in the presence of 10⁻⁷M Tm, and was split every three to four days. 27 days later, the cells were washed and further cultured in the presence or absense of Tm. MTS assays were performed as described below.

FIG. 24 shows expression of CD34 in the bone marrow cells into which “ΔGCR-Mpl-TmR-IRES-EGFP” has been introduced, detected by flow cytometry.

FIG. 25 shows the construction of MSCV-EPORwt.

FIG. 26 shows the construction of MSCV-EPORMpl and MSCV-EPORGCR.

FIG. 27 shows the construction of MSCV-EPORwt (or EPORMpl or EPORGCR)-IRES-mitoYFP.

FIG. 28 shows the dose-dependency of the EPO-driven SAG-transduced Ba/F3 cells.

FIG. 29 shows that the EPO-driven SAGs showed higher levels of proliferation compared to the tamoxifen(Tm)-driven SAG

FIG. 30 shows the expansion rate of transduced Ba/F3 cells

FIG. 31 shows that removal of EPOR decreases the number of cells.

FIG. 32 shows the transduction of human cord blood CD34+ cells.

FIG. 33 shows the number of c-kit-positive cells.

FIG. 34 shows the In vitro clonogenic progenitor assay.

Best Mode for Implementing the Invention

### EXAMPLE 1

### Constructing the Chimeric G-CSF Receptor/Estrogen Receptor Gene (a Selective Amplification Gene)

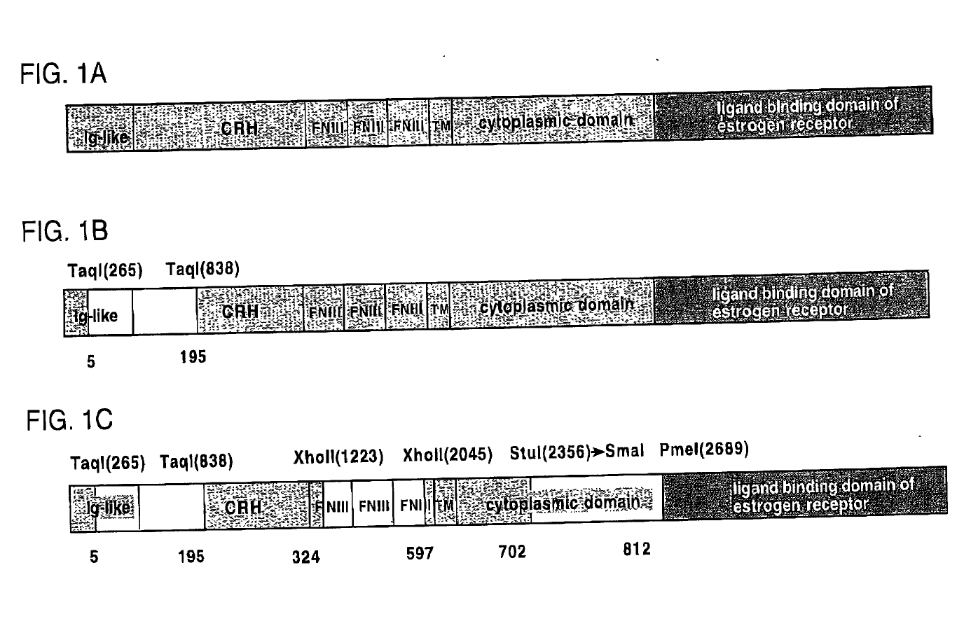

In order to produce a chimeric protein comprising the entire G-CSF receptor and the ligand (estrogen)-binding domain of the estrogen receptor (hereafter designated simply as “GCRER”), the fusion gene having cDNAs that encode the respective proteins (FIG. 1(A)) was constructed. Next, a mutant of the fusion gene, “GCRER,” which is deficient in the 5th residue, Glu, through the 195th residue, Leu, of the G-CSF receptor extracellular domain (hereafter designated simply as “GCRΔ(5-195)/ER”) was constructed, in order to produce a chimeric protein that lacks reactivity against G-CSF (FIG. 1(B)). Further, a mutant was constructed by deleting a portion containing the differentiation-inducing domain (725-756) of the G-CSF receptor from the mutant (hereafter designated simply as “GCRΔ(5-195, 725-756)/ER”) (FIG. 1(C)).

### EXAMPLE 2

### Isolation of Ba/F3 Cells into Which was Introduced the Chimeric G-CSF Receptor/Estrogen Receptor Gene, Which is a Selective Amplification Gene

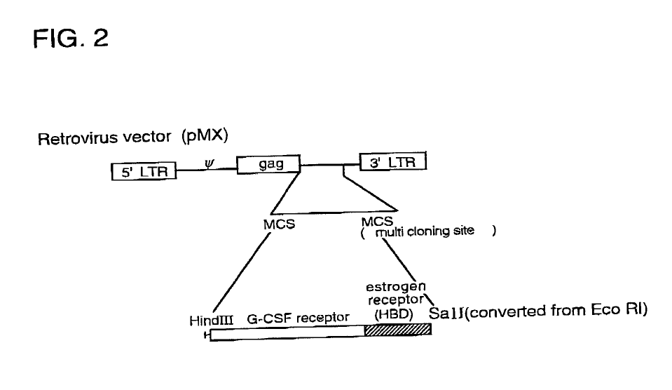

The three kinds of selective amplification genes prepared in Example 1 were introduced into plasmid “PCMX” (Cancer Res. 56:4164 (1996)). Ten μg each of the resulting plasmids were introduced into the Ba/F3 cell, which is an IL-3-dependent cell line, together with 1 μg of the ScaI-linearized “pSV2bsr” (Kaken Pharmaceuticals) carrying a blasticidin resistance gene, by electroporation. After the electroporation, the cells were distributed into 24-well plates at 5×10⁵ cells per well, and cultured in a medium containing 10 μg/ml of blasticidin. Proliferation of blasticidin resistant cells was observed in 11 out of 17 wells where “pCMX-GCRER” was introduced, in 3 out of 29 wells where “PCMX-GCRΔ(5-195)/ER” was introduced, and in 52 out of 52 wells where “pCMX-GCRΔ(5-195, 725-756)/ER” was introduced. After allowing these blasticidin resistant cells to proliferate in individual wells with IL-3, the cells were cultured with 10⁻⁷ M estradiol instead of IL-3. Proliferation of IL-3-independent and estrogen-dependent cells was observed in 7 out of 11 wells where “PCMX-GCRER” was introduced, in 3 out of 3 wells where “pCMX-GCRΔ(5-195)/ER” was introduced, and in 13 out of 16 wells where “pCMX-GCRΔ(5-195, 725-756)/ER” was introduced. When a similar experiment was performed using, in place of “pCMX-GCRER,” a retrovirus vector “pMX” (Exp. Hematol. 24: 324 (1996)) into which “GCRER” had been inserted (hereafter designated simply as “pMX-GCRER”) (FIG. 2), proliferation of IL-3-independent and estrogen-dependent cells was observed in 2 out of the 24 wells each containing one cell. Also, when 1 nM G-CSF was added in place of estradiol to the cells into which “pCMX-GCRER” was introduced, those wells that showed G-CSF-dependent proliferation were the same as those that had shown estradiol-dependent proliferation. Moreover, when the Ba/F3 cells containing no plasmid were used as a control, neither G-CSF-dependent proliferation nor estradiol-dependent proliferation was observed. The production of the desired fusion protein in the cells was confirmed by western blotting using an anti-G-CSF receptor antibody or an anti-estrogen receptor antibody.

### EXAMPLE 3

### Analysis of Cell Proliferation by Estradiol

Among the clones obtained by limiting dilution in Example 2, those showing good response to estradiol were selected and used in the following experiment (XTT assay).

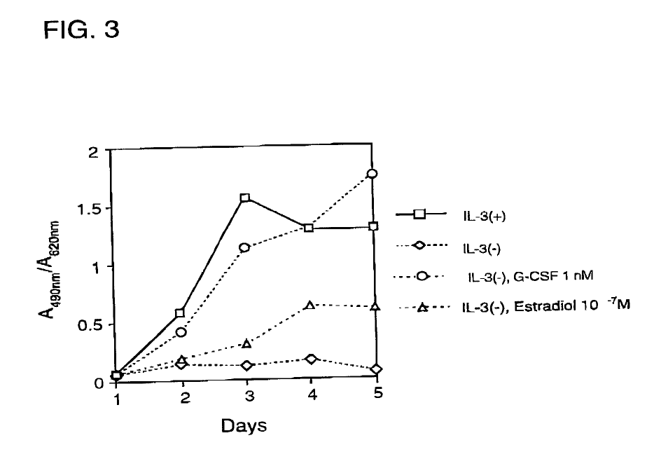

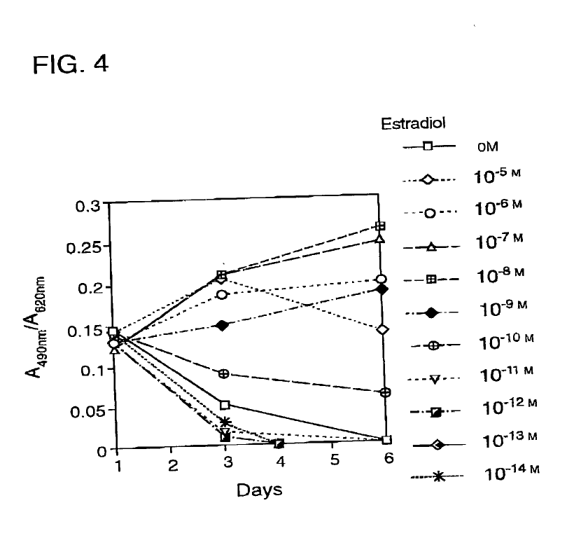

The Ba/F3 cells into which “pCMX-GCRER” was introduced were examined. There were IL-3-independent cells that proliferated by stimulation with G-CSF or estradiol (FIG. 3). Moreover, when the same experiment was done while varying the estradiol concentrations between 10⁻¹⁴ and 10⁻⁷ M, cell proliferation was observed in the range from 10⁻⁹ to 10⁻⁷ M (FIG. 4). This result suggests that estradiol transmits the cell proliferation signal at the concentrations between 10⁻⁹ and 10⁻⁷ M.

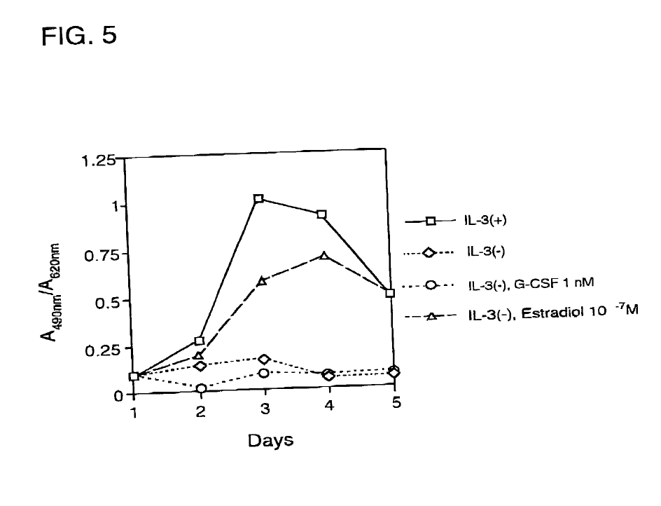

The Ba/F3 cells into which “pCMX-GCRΔ(5-195)/ER” was introduced were then examined. The results indicated that the cell proliferation by G-CSF stimulation was blocked and the estradiol stimulation alone caused cell proliferation (FIG. 5).

Similarly, for Ba/F3 cells into which “pCMX-GRΔ(5-195, 725-756)/ER” was introduced, cell proliferation was caused by estrogen stimulation, but no response to G-CSF was observed.

### EXAMPLE 4

### Construction of the IRES-CD24 Expression Plasmid

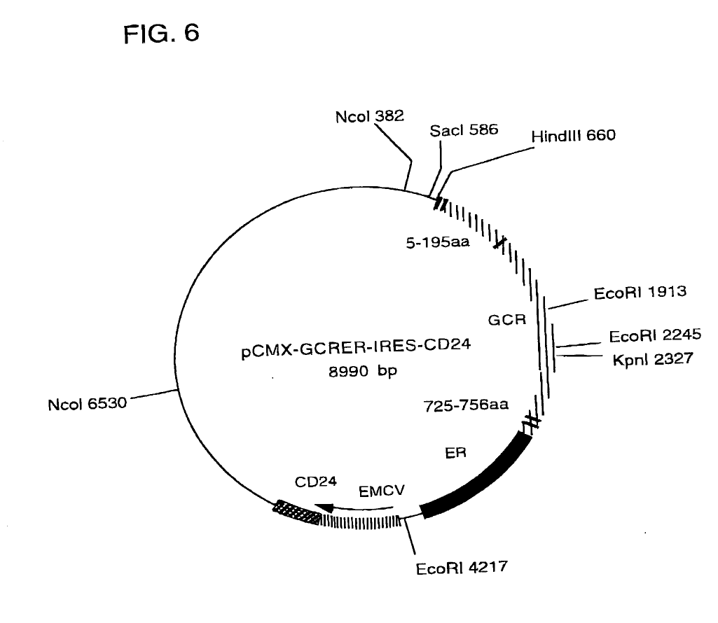

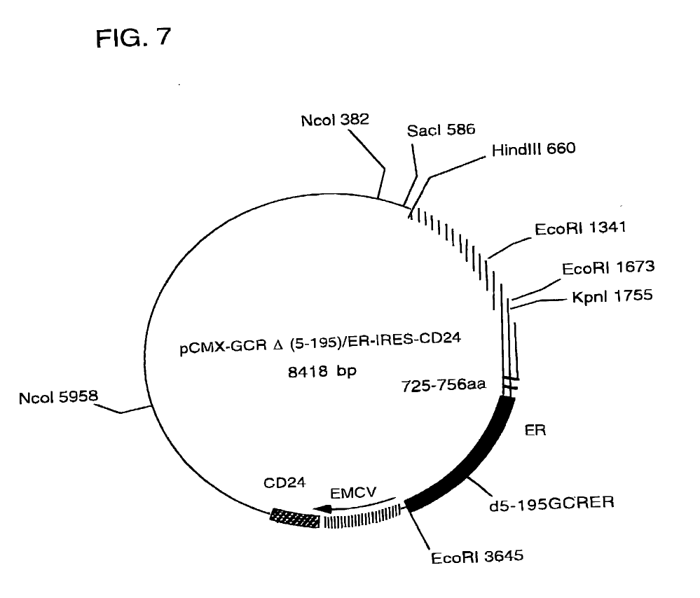

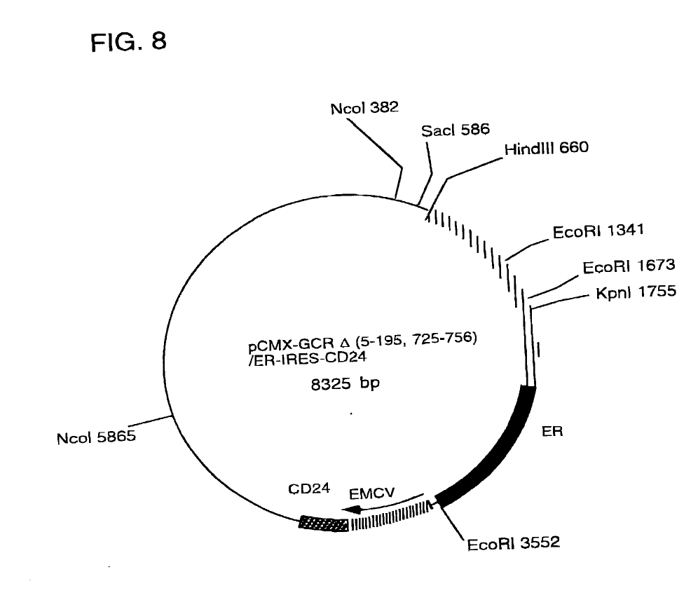

“PCMX-GCRER” was digested with HindIII and EcoRI, and the vector fragment (“fragment 1”) was recovered. Also, from “pCMX-GCRER” and “pCMX-GCRΔ(5-195)/ER,” the HindIII fragment (“fragment 2,” 1672 bp) and the KpnI fragment (“fragment 3,” 1099 bp), and the EcoRI fragment (“fragment 4,” 1888 bp) and the KpnI fragment (“fragment 5,” 1792 bp) were recovered. PBCEC (pBluescript II KS ligated to IRES and CD24 derived from EMCV, Migita, M., Proc. Natl. Acad. Sci. USA 92:12075 (1995)) was digested with ApoI, and the fragment containing IRES-CD24 (“fragment 6,” 950 bp) was recovered. “pCMX-GCRER-IRES-CD24” (FIG. 6) was constructed by ligating “fragment 1,” “fragment 2,” “fragment 4,” and “fragment 6.” “pCMX-GCRΔ(5-195)/ER-IRES-CD24” (FIG. 7) was constructed by ligating “fragment 1,” “fragment 3,” “fragment 4,” and “fragment 6.” “pCMX-GCRΔ(5-195, 725-756)/ER-IRES-CD24” (FIG. 8) was constructed by ligating “fragment 1,” “fragment 3,” “fragment 5,” and “fragment 6.”

### EXAMPLE 5

### Intracellular Expression of CD24

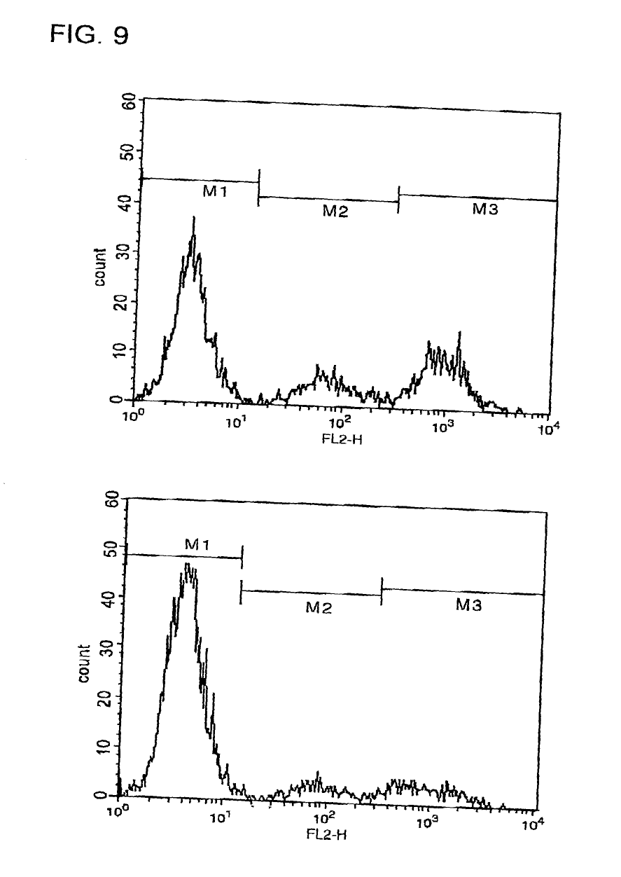

After 10⁷ Ba/F3 cells were washed twice with PBS and once with “OPTI-MEM1” (Gibco-BRL), the cells were suspended into 0.2 ml of “OPTI-MEM1.” Ten mg each of “pCMX-GCRER-IRES-CD24,” “pCMX-GCRΔ(5-195)/ER-IRES-CD24,” and “pMX-GCRΔ(5-195, 725-756)/ER-IRES-CD24” was added to the cells, and transformation was performed using “Gene Pulser” (BioRad) at 290 V, 960 mF. After the transformation, the cells were cultured for two days in the RPMI medium containing 10% FCS and 10 U/ml mIL-3 (R&D SYSTEMS). After 10⁶ cells were washed with 5% FCS/PBS, the cells were reacted with 1 mg/ml of the anti-CD24 antibody (Pharmingen) for 30 minutes at room temperature. The cells were then washed twice with 5% FCS/PBS, reacted with a 1:20 dilution of the PE-labeled anti-mouse antibody (DAKO) for 30 minutes at room temperature, and washed again twice with 5% FCS/PBS. The cells were suspended in 1 ml of 5 mg/ml propidium iodide/PBS, and the CD24 expression was analyzed by flow cytometry (Becton Dickinson) using a 585 nm detector. The CD24 expression was detected from a number of the cells into which “pCMX-GCRΔ(5-195)/ER-IRES-CD24” had been introduced. In this experiment, the cells into which “pCMX-GCRΔ(5-195)/ER” was introduced were used as a control against the cells having “pCMX-GCRΔ(5-195)/ER-IRES-CD24” introduced. The results are shown in FIG. 9 and Table 1. Note that the data contain the signal from propidium iodide that was used to detect the dead cells.

TABLE 1

| | Anti-CD24 antibody | Anti-CD24 antibody |
|---|---|---|
| Introduced plasmid | (−) cells | (+) cells |
| PCMX-GCRΔ (5-195)/ | 59.77% | 40.23% |
| ER-IRES-CD24 | | |
| pCMX-GCRΔ (5-195)/ | 85.10% | 14.90% |
| ER | | |

### EXAMPLE 6

### Progenitor Assays

5-Fluorouracil (5FU: Wako Pure Chemical Industries, Ltd.) in physiological saline (10 mg/ml) was intravenously injected into four 6-week-old C57BL mice at a dose of 330 ml/mouse. Two days after the injection, bone marrow was collected from femurs, centrifuged (1,500 rpm, 25, 22 min) on “Lymphocyte-M” (Cederlane) to isolate mononuclear cells. The mononuclear cells were cultured for two days in the Iscove modified Dulbecco medium (IMDM; Gibco) supplemented with 20% FCS, 100 U/ml IL6, and 100 mg/ml rat SCF. On a CH296 (Takara Shuzo; Hanenberg, H. et al., Nature Med. 2: 876 (1996))-coatedplate (1146: Falcon) 10⁶ bone marrow cells pretreated with IL6 and SCF were suspended in a culture supernatant containing 10⁸ of either the retrovirus “vMXGCRER” (obtained in the culture supernatant of an ecotropic packaging cell line “GP+E-86” (J. Virol. 62: 1120 (1988)) and having “pMX-GCRER” incorporated therein) or the retrovirus “vMXGCRΔ(5-195)/ER” (obtained in the culture supernatant of an ecotropic packaging cell line “GP+E-86” and having “pMX-GCRΔ(5-195)/ER” introduced therein). The cells were cultured in the presence of IL6 and SCF. The viral supernatants were replaced at 2, 24, 26, 36, and 38 hours. Twenty-four hours after the sixth viral supernatant replacement, the cells were transferred into a medium containing methylcellulose (IMDM, 1.2% methylcellulose 1,500 cp; Wako, 20% FCS, 1% deionized BSA, 10 mM2-mercaptoethanol, 10⁻⁷ M b-estradiol) at 10⁴/well. After culturing for 10 days, colonies were observed under the microscope. Smear samples were prepared and subjected to Wright-Giemsa staining to identify the cells.

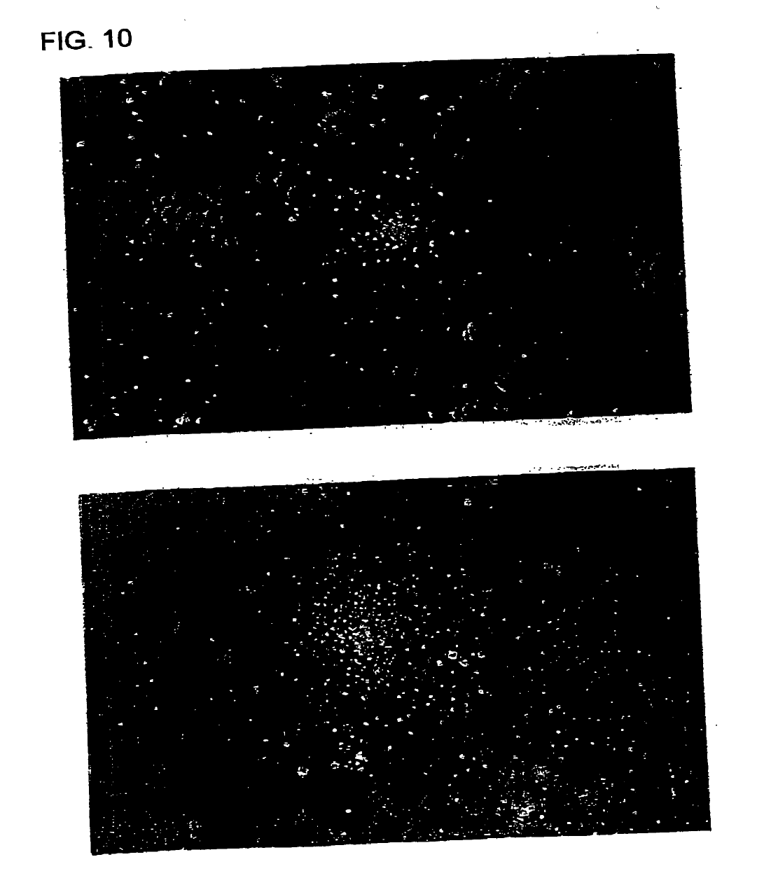

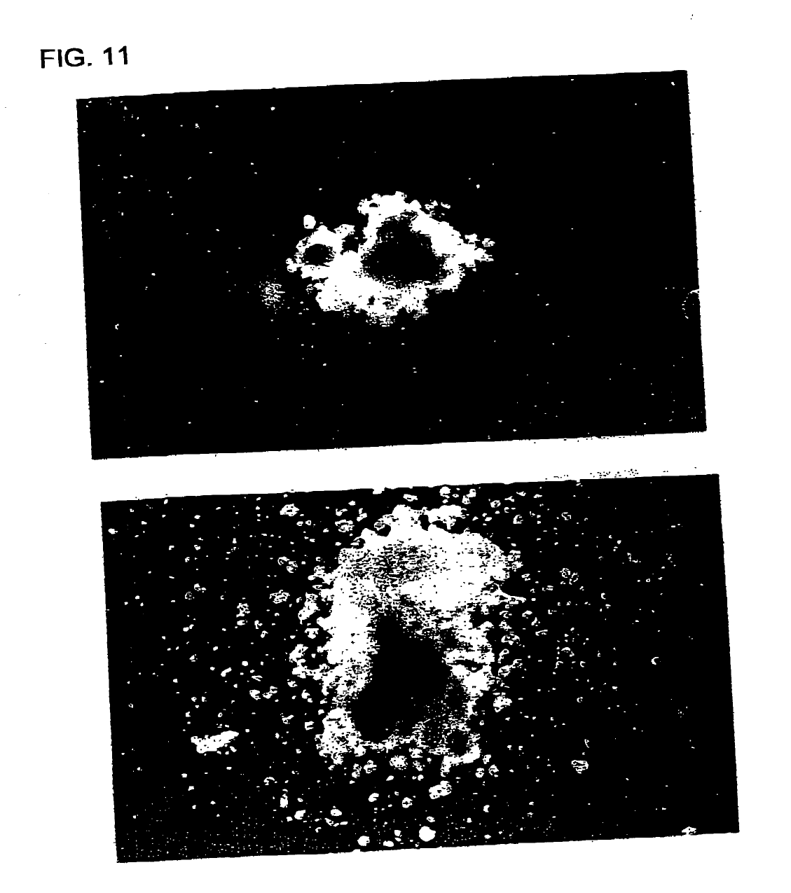

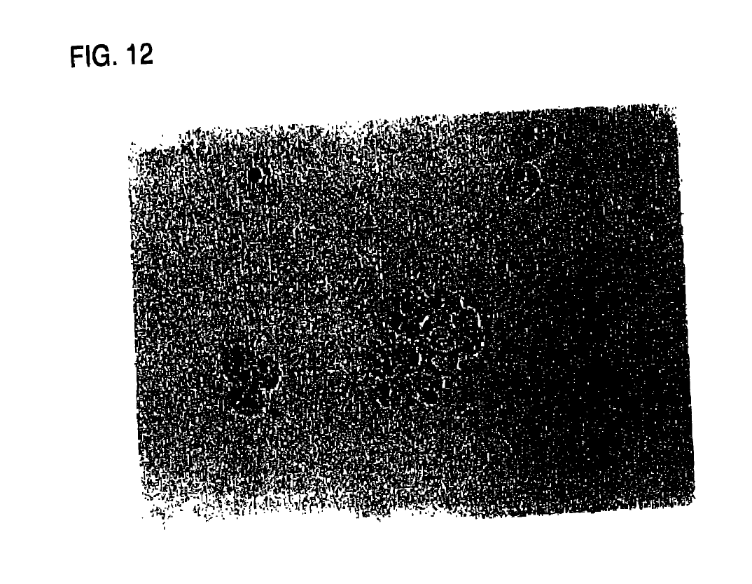

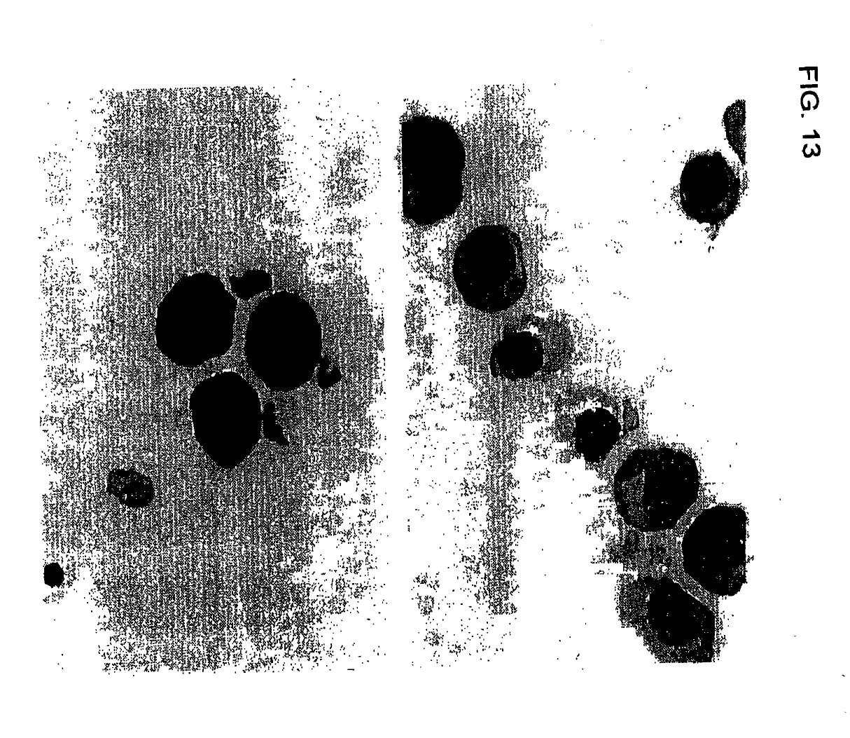

Among the bone marrow cells infected with “vMXGCRER” or “vMXGCRΔ(5-195)/ER,” granulocyte-macrophage lineage colonies and erythroblast lineage colonies, which had differentiated from the bone marrow cells by the estradiol stimulation, were observed. FIG. 10 shows the granulocyte-macrophage lineage colonies derived from the “vMXGCRER”-infected bone marrow cells by the estradiol stimulation; FIG. 11 shows the erythroblast lineage colonies derived from the “vMXGCRΔ(5-195)/ER”-infected bone marrow cells upon the estradiol stimulation. When the cells constituting these colonies were made into smear samples and subjected to Wright-Giemsa staining, differentiated blood cell images were obtained. FIG. 12 shows the Wright-Giemsa stained image of the macrophage observed in the smear samples of the granulocyte-macrophage lineage colonies derived from the “vMXGCRER”-infected bone marrow cells; FIG. 13 shows the Wright-Giemsa stained image of the erythroblasts observed in the smear samples of the erythroblast lineage colonies derived from the “vMXGCRΔ(5-195)/ER”-infected bone marrow cells.

### EXAMPLE 7

### Examination for Ligand-Inducible Growth Using the Selective Amplifier Gene Encoding the Fusion Proteins, GCRTmR and ΔGCRTmR.

(1) Construction and Expression of GCRTmR and ΔGCRTmR Chimera.

A murine IL-3 expression plasmid pBMG-hph-IL3 was constructed for high-titer production of the cytokine by cultured cell's. This plasmid contains a Rous sarcoma virus promoter-driven IL-3 cDNA, and a hygromycin B-resistance gene (from pY3 plasmid) in the pBMGNeo backbone [Gorman C M, et al., Proc. Natl. Acad. Sci. USA., (1982) 79: 6777-6781; Fung M C, et al., Nature, (1984) 307:233-237; Blochlinger K, and Diggelmann H., Mol. Cell. Biol.,(1984) 4: 2929-2931; Karasuyama H, and Melchers F., Eur. J. Immunol., (1988) 18: 97-104].

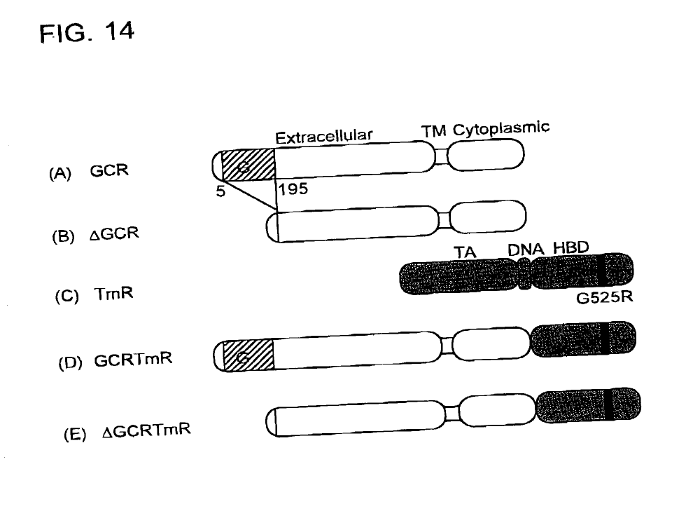

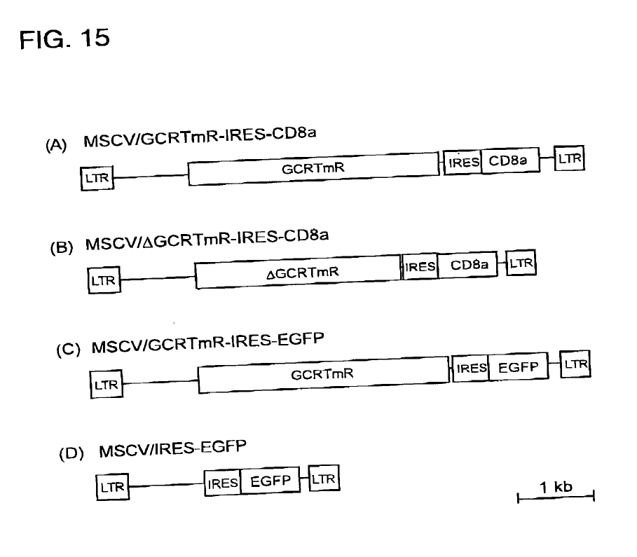

The structures of GCR, ΔGCR, Tm-responsive mutant estrogen receptor (TmR), and the newly constructed selective amplifier gene products (GCRTmR and ΔGCRTmR) are shown in FIG. 14. GCRTmR was a fusion construct between the full-length mouse GCR and the G52SR mutant murine ER, which was expected to bind G-CSF and Tm to transmit growth signals. As a derivative, ΔGCRTmR was constructed by deleting the G-CSF-binding domain (amino acids 5-195) from GCRTmR. We cloned the GCRTmR and ΔGCRTmR cDNAs to make bicistronic retrovirus vectors which also harbored the murine CD8a gene as a second cistron (MSCV/GCRTmR-IRES-CD8a and MSCV/ΔGCRTmR-IRES-CD8a; FIG. 15).

To transduce the modified selective amplifier genes into hematopoietic cells, bicistronic expression vectors were constructed as follows. The murine phosphoglycerate kinase promoter-neomycin phosphotransferase gene cassette (EcoR I-Sal I) in MSCV2.2 retrovirus (a gift from Dr. R. G. Hawley, University of Toronto, Canada) was replaced with the murine CD8a cDNA under control of the encephalomyocarditis virus (EMCV)-derived internal ribosome entry site sequence (IRES; nucleotides 259-833 of EMCV-R genome) to construct MSCV/IRES-CD8a [Hawley R G, et al., Gene Ther.,(1994) 1: 136-138; Nakauchi H, et al., Proc. Natl. Acad. Sci. USA., (1985) 82: 5126-5130; Duke G M, et al., J. Virol., (1992)-66: 1602-1609]. The murine cDNAs encoding GCR and ΔGCR were obtained as BamH I-Pme I fragments from pMX/GCRER and pMX/ΔGCRER respectively [Ito K, et al., Blood, (1997) 90:3884-3892]. TmR cDNA was derived from pBS+ER T (a gift fromDrs. T. D. Littlewood and G. I. Evan, Imperial Cancer Research Fund, London, UK) [Danielian P S, et al., Mol. Endocrinol., (1993) 7: 232-240; 17. Littlewood T D, et al., Nucleic Acids Res.,(1995) 23: 1686-1690], by polymerase chain reaction (PCR) with primer A (5′-TAC GTT TAA ACG ATC CGG GCA CTT CAG GAG-3′; SEQ ID NO:1) and primer B (5′-CTG TCG ACA CTA GTA GGA GCT CTC AGA TCG-3′; SEQ ID NO:2), creating a Pme I site in the 5′-end and a Sal I site in the 3′-end [Littlewood T D, et al., Nucleic Acids Res., (1995) 23: 1686-1690; Kodiara H, et al., Jpn. J. Cancer Res., (1998) 89: 741-747]. Along with TmR cDNA, the GCR or ΔGCR was cloned respectively into Bgl II-Xho I site of MSCV/IRES-CD8a by trimolecular ligation, and the resultant vectors were designated as MSCV/GCRTmR-IRES-CD8a and MSCV/ΔGCRTmR-IRES-CD8a.

MSCV/GCRTmR-IRES-EGFP was constructed to transduce primary murine hematopoietic cells by replacing the murine CD8a cDNA in MSCV/GCRTmR-IRES-CD8a with a fragment encoding the enhanced green fluorescent protein (EGFP; derived from pEGFP-1, Clontech, Palo Alto, Calif.). IRES-EGFP without a selective amplifier gene was cloned into MSCV2.2 backbone as a control (MSCV/IRES-EGFP).

To prepare GCRTmR and ΔGCRTmR gene-containing retroviruses, BOSC23 cells were lipofected with MSCV/GCRTmR-IRES-CD8a or MSCV/ΔGCRTmR-IRES-CD8a, and the supernatants were harvested. The viral supernatants were used to infect IL-3-dependent Ba/F3 cells according to a fibronectin-assisted infection procedure [Hanenberg H, et al., Nat. Med., (1996) 2: 876-882].

Ba/F3 cells (Riken Gene Bank RCB0805, Tsukuba, Japan) were maintained in RPMI 1640 (Life Technologies, Grand Island, N.Y.) supplemented with 10% fetal bovine serum (FBS; Bioserum, Victoria, Australia), 100 units/ml (U/ml) penicillin-100 μg/ml streptomycin (Irvine Scientific, Santa Ana, Calif.), and 0.5% conditioned medium of C3H1OT1/2 cells (Riken Gene Bank RCB0247) transfected with pBMG-hph-1L3 as a murine IL-3 source.

Titration studies with mouse BM progenitors revealed that this dose of the conditioned medium had an equivalent titer to 100 U/ml IL-3.

BOSC23 (American Type Culture Collection [ATCC] CRL-11554, Manassas, Va.) and GP+ E86 (kindly provided by Dr. A. D. Miller, Fred Hutchinson Cancer Research Center, Seattle, Wash.) ecotropic packaging cells were maintained in Dulbecco's modified Eagle medium (Life Technologies) containing 10% FBS.

All retroviral transduction experiments were performed in P2 facilities, according to the institutional recombinant DNA biosafety guidelines. BOSC23 cells were transfected with MSCV/GCRTmR-IRES-CD8a or MSCV/ΔGCRTmR-IRES-CD8a using Lipofectamine (Life Technologies) and the viral supernatants were harvested on day two post-lipofection. Fibronectin-assisted transduction of Ba/F3 cells was carried out on 6-well plates precoated with RetroNectin (Takara Shuzo, Otsu, Japan) according to a standard procedure [Hanenberg H, et al., Nat. Med., (1996) 2: 876-882]. After retroviral infection, the transduced Ba/F3 cells were selected with a Magnetic Cell Sorting (MACS) system (Miltenyi Biotec, Bergisch Gladbach, Germany). Aliquots of 1×10⁷ Ba/F3 cells were incubated with anti-CD8a antibody-conjugated microbeads, and CD8a-positive cells were recovered according to the manufacturer's protocol. The selected cells were analyzed for CD8a expression by fluorescence-activated cell sorting (FACS) with a fluorescein isothiocyanate (FITC)-labeled anti-murine CD8a antibody (Pharmingen, San Diego, Calif.) and a FACS can (Becton Dickinson, Palo Alto, Calif.). Successfully transduced Ba/F3 cells (BaF/GCRTmR and BaF/ΔGCRTmR) were cloned by limiting dilution, with an initial incubation with IL-3 for six days followed by expansion with 10⁻⁷ M Tm (Sigma, St. Louis, Mo.).

After transduction, CD8a expression in an aliquot of Ba/F3 cells was analyzed by FACS, and the transduction efficiency was estimated to be between 38% and 54% by counting CD8a-positive cells. The remainder of the cells were subjected to MACS selection, and nearly 100% of the recovered Ba/F3were CD8a-positive. Subsequently, the selected Ba/F3 cells were cloned by limiting dilution; 11 out of 52 isolates of BaF/GCRTmR and nine out of 20 isolates of BaF/ΔGCRTmR showed Tm-responsive growth. Three of these isolates expressing GCRTmR or ΔGCRTmR were randomly chosen for further characterization, and FACS confirmed CD8a expression in these clones.

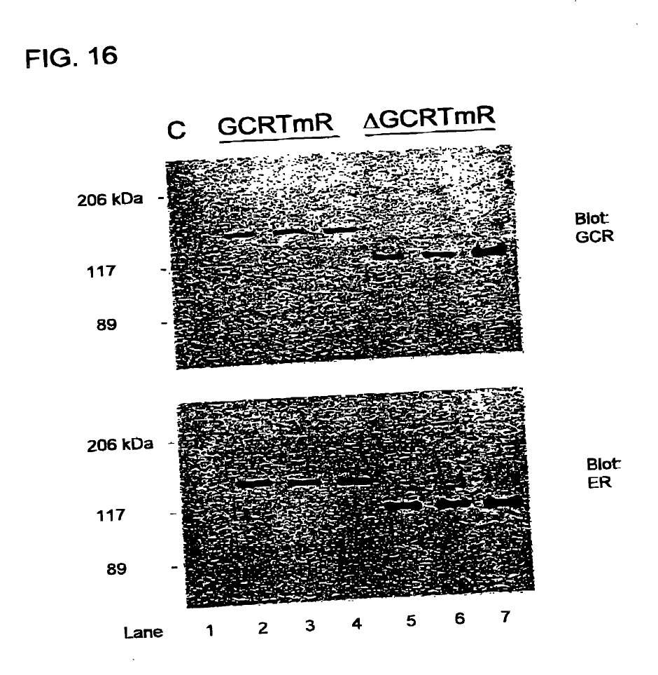

Expression of GCRTmR and ΔGCRTmR in the selected Ba/F3 clones were determined by a western blot as shown in FIG. 16.

Aliquots of 1×10⁷ parental and transduced Ba/F3 cells were lysed with NP-40 lysis buffer (1% NP-40, 150 mM NaCl, 50 mM Tris-HCl [pH 7.4], 500 U/ml aprotinin, and 1 mM phenylmethylsulfonyl fluoride) Protein concentrations of the lysates were determined by BCA Protein Assay (Pierce, Rockford, Ill.). Protein samples (10 μg/lane) were electrophoresed on 7.5% sodium dodecyl sulfate-polyacrylamide gels and electroblotted onto Immobilon-P polyvinylidene fluoride (PVDF) membranes (Millipore, Yonezawa, Japan). After blocking with 4% bovine serum albumin (Boehringer Mannheim), the membranes were incubated with an anti-GCR antibody (M-20; Santa Cruz Biotechnology, Santa Cruz, Calif.) or an anti-ER antibody (MC-20; Santa Cruz Biotechnology). The fusion proteins were visualized by an ECL system (Amersham, Little, Chalfont, UK).

Probing with either an anti-GCR or an anti-ER antibody revealed a peptide of 140 kDa (lanes 2-4) in the GCRTmR-transduced clones, and a 120 kDa protein (lanes 5-7) in the ΔGCRTmR-transduced clones, with the same apparent molecular weights for the fusion proteins GCRER and ΔGCRER described previously [Ito K, et al., Blood, (1997) 90: 3884-3892].

(2) Proliferation of Ba/F3 Expressing GCRTmR or ΔGCRTmR.

Representative BaF/GCRTmR and BaF/ΔGCRTmR clones were stimulated by IL-3, 10⁻⁹ M human G-CSF (rhG-CSF; provided by Chugai Pharmaceuticals, Tokyo, Japan), 10⁻⁷ M Tm or 10⁻⁷ M β-estradiol (E₂; Sigma). Cultures containing IL-3 or G-CSF were split every three to four days, while Tm-containing cultures were diluted every four days. Cell proliferation assay was performed periodically in 96-well microtiter plates by the 2,3-bis (2-methoxy-4-nitro-5-sulfophenyl)-5-[(phenylamino)-carbonyl]-2H-tetrazolium hydroxide (XTT) method using Cell Proliferation Kit II (Boehringer Mannheim, Mannheim, Germany) [Scudiero D A, et al., Cancer Res., (1988) 48: 4827-4833]. For hormone dose-response analysis, transduced Ba/F3 cells were stimulated by various concentrations of Tm or E₂, and XTT assay was performed on day zero and day four. The final concentration of ethanol used to dilute Tm and E₂ was 0.1% in all the culture conditions.

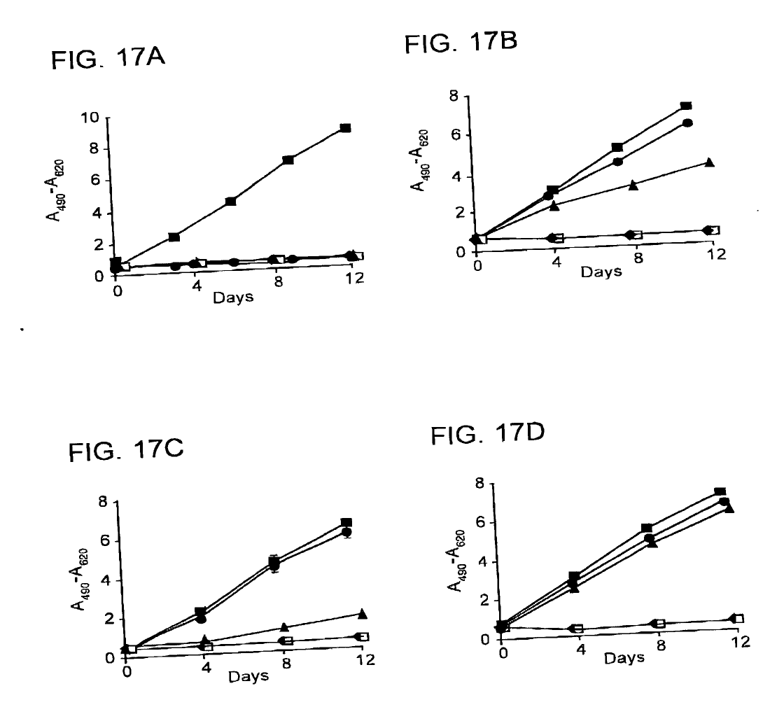

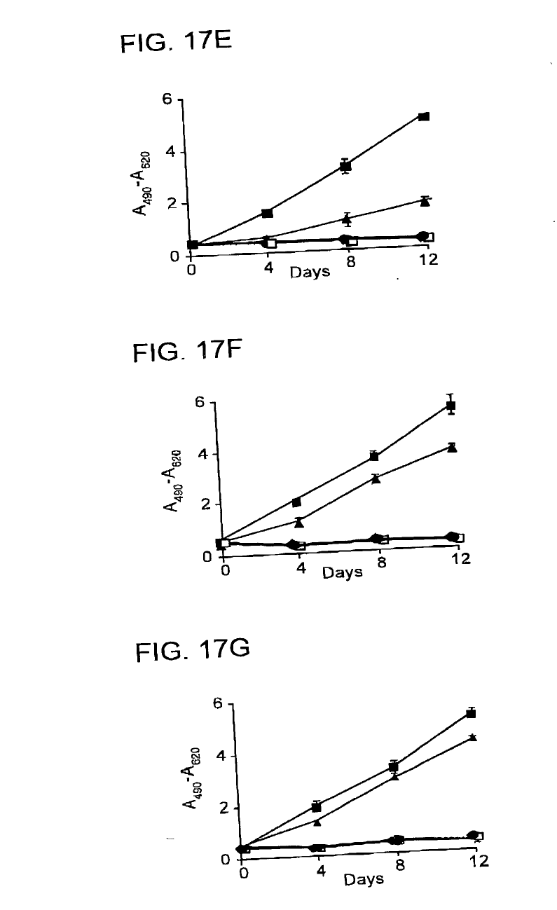

Parental Ba/F3 cells were dependent on IL-3, and all cells died upon its withdrawal. G-CSF, Tm, and E₂ did not support untransduced Ba/F3; switching from IL-3 to any of these stimuli resulted in rapid and extensive apoptosis (FIG. 17A). GCRTmR-transduced Ba/F3 cells proliferated not only with IL-3, but also grew continuously with either 10⁻⁹ M G-CSF or 10⁻⁷ M Tm. The growth rates of the GCRTmR-transduced clones were almost identical regardless of whether the cells were incubated with IL-3 or G-CSF, but their responses to Tm varied as shown in FIG. 17B-D. Among three clones examined, clone 3 showed the greatest growth rate with Tm which was comparable to those with IL-3 and G-CSF. Clone 2 showed the least response and clone 1 showed an intermediate response to Tm. The amount of GCRTmR protein in clone 2 seemed a little less than the other two clones (FIG. 16), which may account for the clonal variation in sensitivity to Tm. In contrast to Tm stimulation, 10⁻⁷ M E₂ had no effect on BaF/GCRTmR clones. In our previous study, E₂ at this dose was optimal to support Ba/F3 cells expressing GCRER or ΔGCRER [Ito K, et al., Blood, (1997) 90: 3884-3892]. Thus, these observations suggested that GCRTmR was selectively activated by Tm while it was inert to E₂. Meanwhile, the BaF/ΔGCRTmR clones did not respond to G-CSFbut proliferated inresponse to Tm (FIG. 17E-G). As was seen in the GCRTmR-transduced Ba/F3, variable sensitivity to Tm was observed among BaF/ΔGCRTmR clones, which may also depend on ΔGCRTmR expression level.

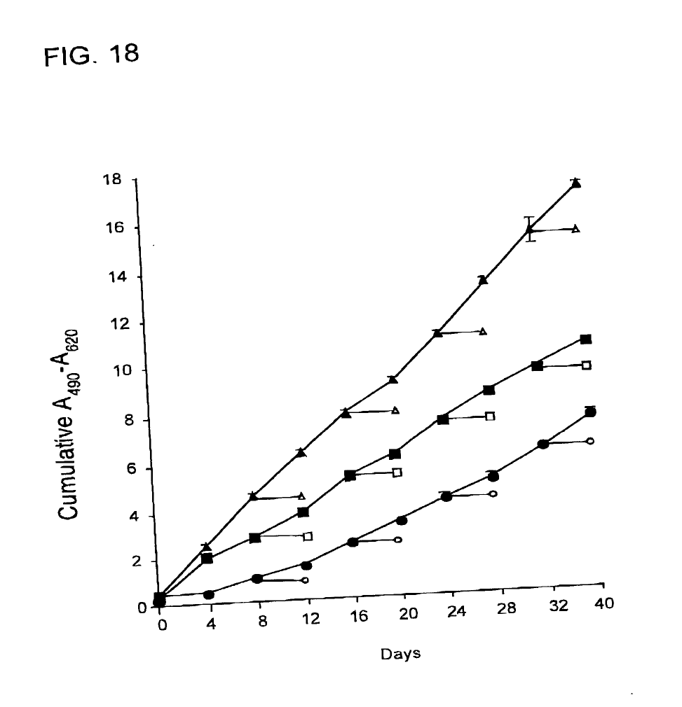

When the BaF/GCRTmR clones were subjected to long-term culture without IL-3, Tm alone could support the cells for at least 36 days, and the cells stopped growing and died within 24 hours upon removal of Tm from the media (FIG. 18). Thus, BaF/GCRTmR cells maintained a Tm-dependence throughout the culture period, and the on/off switching of the growth signal via GCRTMR was effectively controlled by Tm.

(3) Hormone Dose-Dependence of GCRTmR-Mediated Growth.

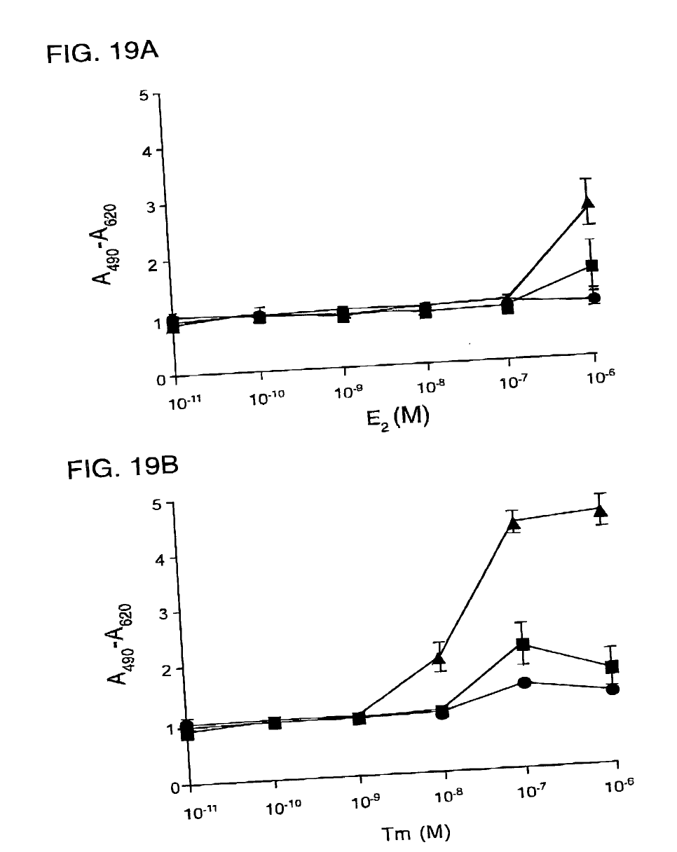

To determine the specificity of GCRTmR for Tm against E₂, growth rates of BaF/GCRTmR clones were examined at various concentrations of Tm and E₂ (FIG. 19). BaF/GCRTmR clone 3 partially responded to 10 M Tm, and all the three clones grew well with Tm at 10⁻⁷-10⁻⁶ M. In contrast, these clones were refractory to estrogen; no growth was observed with up to 10⁻⁷ M E₂. Clone 3 showed a limited response to 10⁻⁶ M E₂, while the other clones were inert with this dose of E₂. Thus, GCRTmR-expressing cells appeared to be at least 100-fold more sensitive to Tm against E₂. Since E₂ induced proliferation of GCRER-expressing Ba/F3 at 10⁻¹⁰ M or greater concentrations [Ito K, et al., Blood,(1997) 90: 3884-3892], GCRTmR-expressing Ba/F3 cells were at least 1000-fold more resistant to E₂ than GCRER-expressing cells. When E₂ or Tm was added to the parental and transduced Ba/F3 cultures with IL-3, neither reagent showed toxic effects at concentrations up to 10⁻⁶ M. Taken together, the optimal concentration of Tm to stimulate BaF/GCRTmR appeared to be 10⁻⁷-10⁻⁶ M.

(4) Clonogenic Progenitor Assay of Transduced Murine Bone Marrow Cells.

BM cells from 5-FU-treated mice were transduced respectively with retrovirus vectors containing the genes for EGFP, GCRER, GCRTmR and ΔGCRTmR.

GCRTmR, ΔGCRTmR and EGFP retroviral supernatants were prepared by transfecting BOSC23 cells as described above. GCRER-viral supernatant was obtained from a selected GP+E86/GCRER producer clone [Ito K, et al., Blood,(1997) 90: 3884-3892]. Six-week-old C57BL/6 mice (purchased from Clea, Tokyo, Japan) were injected intraperitoneally with 150 mg/kg 5-fluorouracil (5-FU; F. Hoffmann-LaRoche, Basel, Switzerland), and BM cells were flushed from femora and tibiae two days later. The cells were prestimulated with 100 ng/ml recombinant rat stem cell factor (rrSCF; kindly provided by Amgen, Thousand Oaks, Calif.) and 100 U/ml recombinant human IL-6 (rhIL-6; kindly provided by Ajinomoto, Yokohama, Japan) in α-MEM containing 20% FES at a starting density of 2×10⁶ cells/ml. After 48 hours of prestimulation, BM cells were incubated (5×10⁵ cells/ml) in the viral supernatants on Retro-Nectin-coated plates in the presence of 100 ng/ml rrSCF and 100 U/ml rhIL-6 for three days, with the vector-containing media changed five times. Mock transduction was similarly performed without using a viral supernatant.

The transduced and untransduced BM cells were harvested, and plated onto Petri dishes at 1×10⁵ cells/dish with 1 ml StemPro medium (Life Technologies) containing 10⁻⁹ M rhG-CSF, 10⁻⁷ M Tm, 10⁻⁷ M E₂, or without stimulator. After ten days of incubation at 37 in a humidified atmosphere of 5% C0₂ in air, colonies were scored. EGFP expression was examined with an inverted fluorescence microscope (IX70; Olympus, Tokyo, Japan).

Table 2 summarizes the clonogenic progenitor assay.

TABLE 2

| | Number of Colonies^{a} | | | |
|---|---|---|---|---|
| Transgene | Tm | E₂ | None | G-CSF |
| GCRER | 68.0 ± 12.2 | 100.2 ± 7.2 | 19.3 ± 6.1 | 184.7 ± 11.5 |
| GCRTmR | 20.2 ± 1.9 | 0 | 0 | 182.7 ± 15.3 |
| ΔGCRTmR | 52.0 ± 15.5 | 0.7 ± 0.9 | 0.3 ± 0.5 | 136.5 ± 11.2 |
| EGFP^{b} | 0 | 0 | 0 | 108.7 ± 2.1 |
| None | 0 | 0 | 0 | 194.0 ± 13.0 |

^{a}Each value represents mean ± SD of hexaplicate determinants.
^{b}EGFP: enhanced green fluorescent protein.

When stimulated by G-CSF, 100-200 colonies were observed in every culture dish, no matter what supernatant was used during transduction. Untransduced or EGFP-transduced BM cells yielded no colony with E₂ or Tm alone. GCRER-transduced BM cells gave rise to about 100 colonies in response to E₂, 60-80 colonies with Tm, and even about 20 colonies were formed without any stimulator. Comparable background colonies were observed in our previous study [Ito K, et al., Blood, (1997) 90: 3884-3892], implying nonspecific activation of GCRER by some estrogen-like substances in the media. In contrast, GCRTmR- and ΔGCRTmR-transduced BM showed very strict responses. GCRTmR-transduced BM gave rise to about 20 colonies out of 1×10⁵ cells in the presence of 10⁻⁷ M Tm, but absolutely no colonies were formed with 10⁻⁷ M E₂ or without a stimulator. ΔGCRTmR-transduced BM yielded about 50 colonies with Tm, while minimum number of colonies (less than one out of 1×10⁵ cells) were formed in the dishes with E₂ or no stimulator. These results clearly demonstrated that TmR-containing chimeric receptors (GCRTmR and ΔGCRTmR) transmitted growth signals very specifically in response to Tm, with negligible activation by E₂ in our culture setting. Morphologically, the Tm-induced colonies were mostly myeloid and mixed colonies, including a few erythroid ones; this result paralleled our previous finding on E₂-induced colonies derived from GCRER- and ΔGCRER-transduced BM [Ito K, et al., Blood, (1997) 90: 3884-3892].

When BM cells were infected by MSCV/GCRTmR-IRES-EGFP retrovirus and subjected to progenitor assay, a total of 121 colonies were formed out of 6×10⁵ cells in response to 10⁻⁷ M Tm. Most of the Tm-responsive colonies fluoresced when observed with an inverted fluorescence microscope, indicating that the transduced progenitors expressed both GCRTmR and EGFP trans-genes during colony formation.

### EXAMPLE 8

### Examination for Ligand-Inducible Growth Using the Selective Amplifier Gene Encoding the Fusion Proteins, ΔGCR-Mpl-ER and ΔGCR-Mpl-TmR.

(1) Plasmid Construction

All enzymes used were purchased from New England Biolabs Inc (Beverly, Mass.). A mammalian expression vector pCMX-MfasER (kindly provided by Dr A. Kakizuka, Kyoto University, Kyoto, Japan), which contains the sequence encoding the HBD of rat estrogen receptor (ER) was digested with BamHI and EcoRI. The BamHI-EcoRI fragment containing the ER-HBD was separated by agarose gel electrophoresis and electroelution. This fragment was subcloned into BamHI-EcoRI site of the plasmid pBluescript (TOYOBO, Japan) by ligation. Further, tamoxifen receptor (TmR) cDNA was derived from a retroviral vector MSCV-ΔGCRTmR-IRES-EGFP (kindly provided by Dr R. Xu, Jichi Medical School, Tochigi, Japan) which contains the sequence encoding the HBD of mouse TmR, by polymerase chain reaction (PCR) with primer A (5′-CTGGATCCGGGCACTTCAGGAGAC-3′; SEQ ID NO:3, creating a BamHI site) and primer B (5′-CTGTCGACCACTAGTAGGAGCTCTCA-3′; SEQ ID NO:4, creating a SalI site). This cDNA was subcloned into BamHI-SalI site of the pbluescript by ligation. On the other hand, a mammalian expression vector pcDNA3.1-c-mpl (kindly provided by Dr M. Takatoku, Jichi Medical School, Tochigi, Japan) which contains the cDNA for human c-mpl between the EcoRI and XbaI sites was digested with EcoRI and SacI. The EcoRI-SacI fragment containing most of the extracellular domain of c-mpl was separated by agarose gel electrophoresis and electroelution. Further, the rest c-mpl cDNA between SacI site and the c-terminal cytoplasmic domain was constructed by PCR using the pcDNA-c-mpl as a template with primer C (5′-CCCACCTACCAAGGTCCCTGG-3′; SEQ ID NO:5) and primer D (5′-CGGGATCCAGAGGCTGCTGCCAATAG-3′; SEQ ID NO:6, creating a BamHI site). Then, the murine phosphoglycerate kinase (pgk) promoter-neomycin phosphotransferase gene (neo) cassette (EcoRI-BamHI) in MSCV2.2 retrovirus (a gift from Dr. R. G. Hawley, University of Toronto, Canada) was replaced with the EcoRI-SacI fragment of c-mpl and the SacI-BamHI fragment of c-mpl by trimolecular ligation to construct MSCV-mpl. The pBluescript-ER and the pBluescript-TmR were digested with BamHI and SalI, and the ER and TmR fragments were separated by agarose gel electrophoresis and electroelution. These fragments were cloned into BamHI-SalI site of MSCV-mpl by ligation. The resultant vectors were designated as MSCV-mpl-ER or MSCV-mpl-TmR.

MSCV-ΔGCRmpl-ER and MSCV-AGCRmpl-TmR were constructed as follows. MSCV-ΔGCR-ER (kindly provided by Dr. KM Matsuda, Jichi Medical School, Tochigi, Japan), in which the GCR binding domain was deleted, was digested with HindIII and KpnI, and the HindIII-KpnI fragment containing a part of the ΔGCR was separated by agarose gel electrophoresis and electroelution. The ΔGCR cDNA between KpnI site and the transmembrane region was constructed by PCR using the MSCV-ΔGCR-ER as a template with primer E (5′-GAGTGGGTACCTGAGGCCCCTAGG-3′; SEQ ID NO:7) and primer F (5′-AACTCGAGGCAGCAGAGCCAGGTCAC-3′; SEQ ID NO:8, creating a XhoI site).On the other hand, the cDNA containing the cytoplasmic region of c-mpl was constructed by PCR using the pcDNA-c-mpl as a template with primer G (5′-AACTCGAGAGGTGGCAGTTTCCTGCA-3′; SEQ ID NO:9, creating a XhoI site) and primer D. The extracellular region of ΔGCR and the cytoplasmic region of c-mpl were cloned into HindIII-BamHI site of pEGFP-Nl (Clontech, Palo Alto, Calif., USA) by ligation (pEGFP-ΔGCRmpl). Then, pgk and neo cassette (BglII-SalI) in MSCV2.2 were replaced with the BglII-BamHI fragment containing the ΔGCRmpl and the BamHI-SalI fragment containing the ER or the TmR. The resultant constructs were designated as MSCV-ΔGCRmpl-ER or MSCV-ΔGCRmpl-TmR.

MSCV-ΔGCRmpl-TmR-IRES-EGFP was constructed as follows. MSCV-ΔGCR-TmR-IRES-EGFP was digested with HpaI and ClaI, and the HpaI-ClaI fragment containing IRES (internal ribosome entry site)-EGFP was separated by agarose gel electrophoresis and electroelution. Then, pgk and neo cassette (BglII-SalI) in MSCV 2.2 was replaced with this fragment. The resultant constructs MSCV-IRES-EGFP was digested with XhoI and ClaI, and the XhoI-ClaI fragment containing IRES-EGFP was separated by agarose gel electrophoresis and electroelution. This fragment was cloned into SalI-ClaI site of MSCV-ΔGCRmpl-TmR by ligation, and the resultant constructs was designated as MSCV-ΔGCR-TmR-IRES-EGFP. All these constructs were confirmed by sequence analysis.

(2) Cell Proliferation Assay

Since TPO is known to stimulate the growth of not only the megakaryocyte lineage but also primitive hematopoietic cells, the intracellular signals from Mpl may be appropriate for selective amplification of transduced HSC.

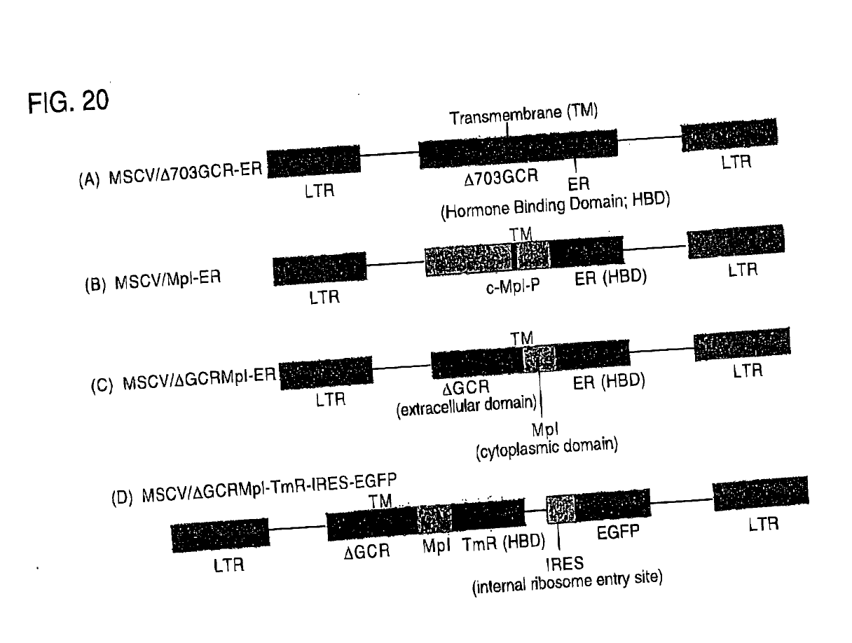

Structures of the vectors used in this study were schematically shown in FIG. 20. BOSC23 cells were transfected with MSCV/ΔGCR-ER orMSCV/Mpl-ER or MSCV/ΔGCR-Mpl-ER or MSCV/ΔGCR-Mpl-TmR-IRES-EGFP using Transfection MBS Mammalian Transfection Kit (Stratagene) and the viral supernatants were harvested on day two post-transfection. Fibronectin-assisted transduction of IL3-dependentBa/F3 cells was carried out on 6-well plates precoated with CH296 fibronectin fragment (Retronectin; Takara Shuzo) according to a standard procedure. After retroviral infection, ΔGCR-Mpl-TmR-IRES-EGFP transduced Ba/F3 cells (BaF/ΔGCR-Mpl-TmR) were selected with EPICS Elite ESP Cell Sorter. GFP-positive cells were removed and were cloned by limiting dilution with 10⁻⁷ M 4-hydroxytamoxifen (Tm).

A quantity of 4×10³ untransduced or transduced Ba/F3 cells in 100 μl was cultured in the presence or absence of 1 ng/ml rmIL-3, 100 ng/ml recombinant human thrombopoietin (rhTPO), or 10⁻⁷M β-estradiol (E2; Sigma), or 10⁻⁷M Tm in 96-well microtiter plates. Cell proliferation assay was periodically performed using CellTier 96 Aqueous One Solution Cell Proliferation Assay {3-(4,5-dimethylthiazol-2-yl)-5-(3-carboxymethoxyphenyl)-2-(4-sulfophenyl)-2H-tetrazolium (MTS); Promega, Madison, Wis.} essentially according to the manufacturer's instruction. In brief, 20 μl per well of MTS-labeling mixture was added. After the incubation at 37° C. for 2 hours, the spectrophotometeical absorbance was measured at the wave length of 490 nm and 650 nm.

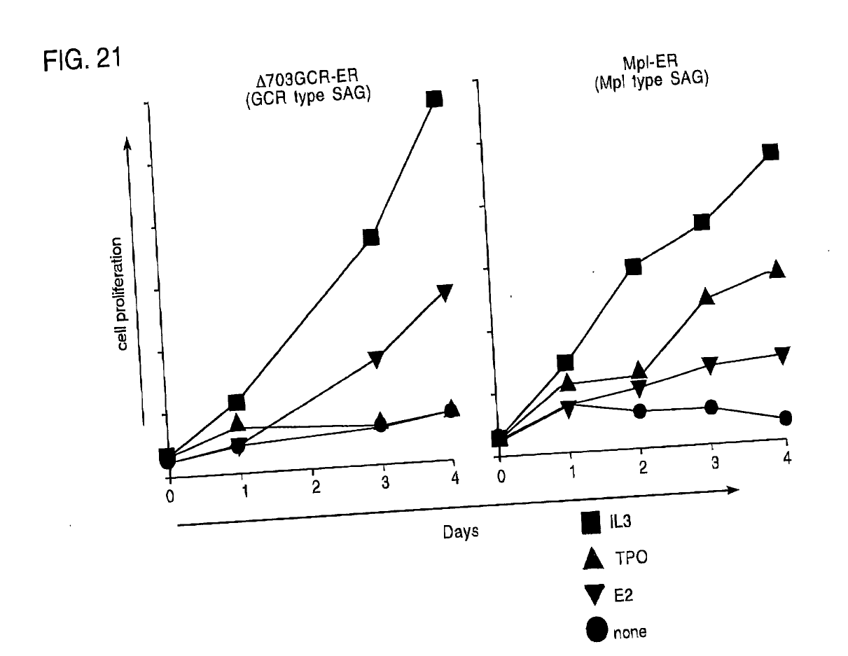

Mpl-mediated signals have been employed in selective amplifier genes. When Mpl-ER chimera was expressed in the interleukin-3 (IL-3)-dependent Ba/F3 cells, the cells acquired the ability to proliferate in response to both estrogen and TPO in the absence of IL-3 (FIG. 21).

However, attempts to minimize the effects of serum TPO by deleting the extracellular domains of Mpl resulted in a total loss of the response to estrogen as well as TPO.

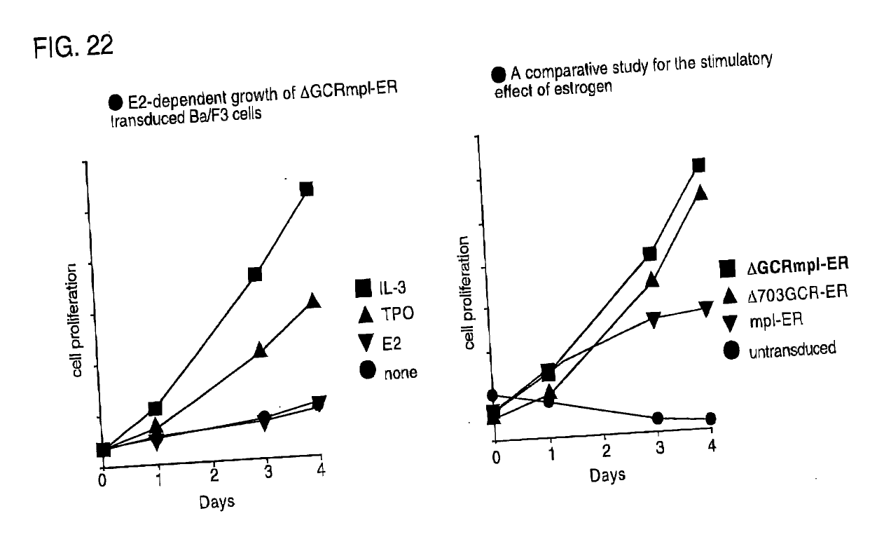

To solve this issue, the extracellular portion of Mpl-ER was replaced with that of ΔGCR-Mpl-ER. Transduction of Ba/F3 cells with the ΔGCR-Mpl-ERgene conferred an estrogen-dependent growth ability on Ba/F3, while the cells were unresponsive to G-CSF or TPO (FIG. 22).

A comparative study for the stimulatory effect of estrogen suggested that the Ba/F3 cells expressing ΔGCR-Mpl-ER proliferated better than the cells expressing ΔGCR-ER or Mpl-ER.

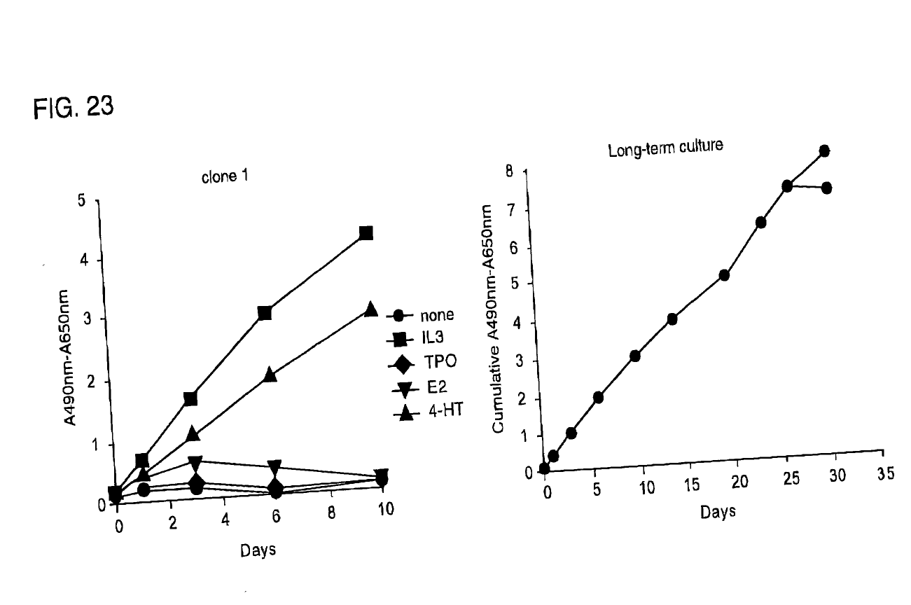

To eliminate the effects of endogenous E2, ER was replaced with a mutant receptor (TmR) which specifically binds to Tm. ΔGCR-Mpl-TmR-expressing Ba/F3 cells showed Tm-dependent growth, while the cells were unresponsive to E2 (FIG. 23).

(3) Transduction of Murine Bone Marrow (BM) Cells

ΔGCR-ER, mpl-ER and ΔGCRmpl-ER retroviral supernatants were prepared by transfecting BOSC 23 cells as described above. Six-week-old C57BL/6 mice (purchased from Clea, Tokyo, Japan) were injected intraperitoneally with 150 mg/kg 5-fluorouracil (5-FU; F. Hoffmann-La Roche, Basel, Switzerland), and 2 days later, BM cells were flushed from femora with IMDM containing 5% FBS. The BM cells were collected by density centrifugation using Lympholyte-M (Cedarlane). Approximately 5×10⁶/ml BM cells were prestimulated with 100 ng/ml of recombinant murine SCF (rmSCF; Pepro Tech Inc, London, England) and 100 U/ml of recombinant human IL-6 (rmIL-6; kindly provided by Ajinomoto, Yokohama, Japan) in IMDM (GIBCO-BRL) containing 20% FBS at 37° C. for 48 hours in a humidified atmosphere of 5% CO2 in air. Subsequently, the cells were resuspended in 2 ml of viral supernatant containing rmSCF and rhIL-6 at a concentration of 5×10⁵ cells/ml, transferred to the 6-well plates precoated with 20 μg/cm² of CH296, and then incubated at 37° C. in a humidified atmosphere of 5% CO₂ in air for 72 hours. During this transduction period, viral supernatant was changed five times. Mock transduction was similarly performed without using a viral supernatant.

(4) In Vitro Clonogenic Progenitor Assay

The transduced and untransduced BM cells were harvested, and 1×10⁵ cells each were plated in 35-mm dish with 1 ml StemPro medium (GIBCO-BRL) in the presence or absence of 100 ng/ml of rhTPO or 10⁻⁷ M of E2. In some experiments, 100 ng/ml of rmSCF, 100 U/ml of rhIL-6, 100 ng/ml of rhTPO, and 2 U/ml of recombinant human erythropoietin (rhEpo; Chugai Pharmaceutical) were added to the cultures. After 10 days of incubation at 37° C. in a humidified atmosphere of 5% CO₂ in air, colonies were scored using an inverted microscope.

(5) Isolation of Cynomolgus CD34-Selected Bone Marrow Cells (BMCs)

Clinically healthy adult cynomolgus monkeys born and reared in Tsukuba Primate Center for Medical Science, National Institute of Health, were used for experiments. BMCs were harvested from femora and suspended in lysis buffer to dissolve red blood cells. Immunomagnetic selection of the CD34+ cells was accomplished using Dynabeads system (Dynal AS, Oslo, Norway) according to the manufacturer's instruction.

(6) Transduction of CD34-Selected BMCs

293T cells were co-transfected with MSCV-ΔGCRmpl-TmR-IRES-EGFP and pCL-Ampho using Transfection MBS Mammalian Transfection Kit (Stratagene, La Jolla, Calif.) and the viral supernatants were harvested on day two and three post-transfection. Cynomolgus CD34-selected cells were placed in 6-well plates precoated with 20 μg/cm² of CH296 and were cultured for 24 hours at 37° C. with 5% CO₂ in Iscove's Dulbecco's medium (IMDM) supplemented with 10% FBS, 50 ng/ml rhIL6, 100 ng/ml rhSCF, 100 ng/ml rhFlt-3 ligand (Research Diagnostic Inc) and 100 ng rhTPO. Subsequently, the cells were resuspended in 1 ml of viral supernatant containing all cytokines described above at a concentration of 1×10⁵ cells/ml. During this transduction period, viral supernatant was changed six times. Mock transduction was similarly performed without using a viral supernatant.

(7) Suspension Culture and Flow Cytometry

After retroviral transduction, CD34-selected BMC were washed and cultured in IMDM supplemented with 10% FBS, 50 U/ml penicillin-50 ug/ml streptomycin, containing 100 ng/ml hrFlt-3 ligand, 10⁻⁷M Tm or without stimulator. On the day 14, aliquots of cells were removed from the suspension culture and tested by flow cytometry.

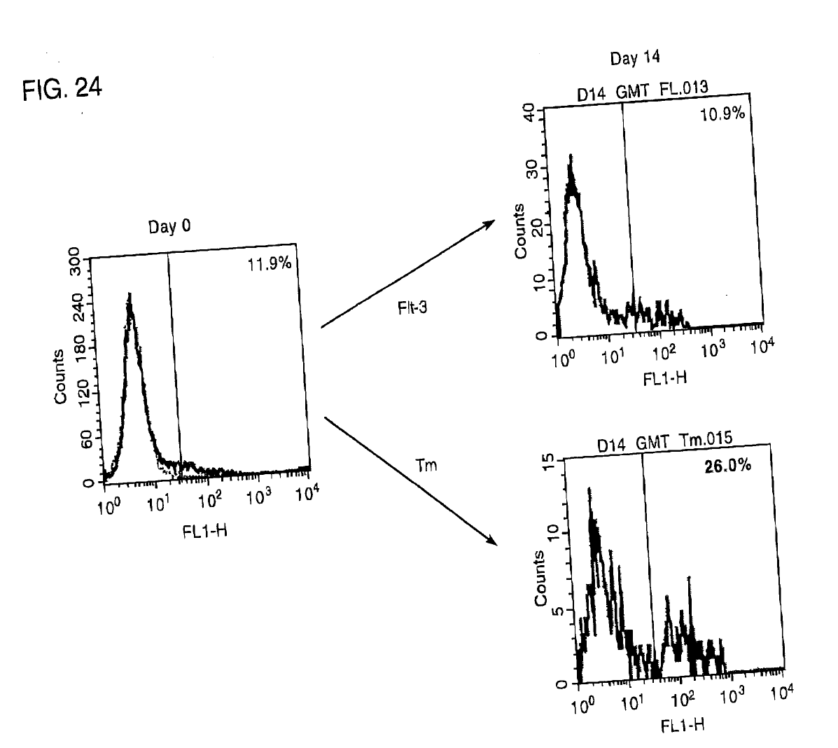

When cynomolgus monkey CD34-selected bone marrow cells transduced with ΔGCR-Mpl-TmR-EGFP were cultured in the presence of Tm, the ratio of GFP of the cells increased about 3-fold relative to the cells cultured in the absence of Tm (FIG. 24).

### EXAMPLE 9

### Examination for Ligand-Inducible Growth Using the Selective Amiplifier Gene Encoding the Fusion Proteins, EPORMpl and EPORGCR.

(1) Plasmid Construction

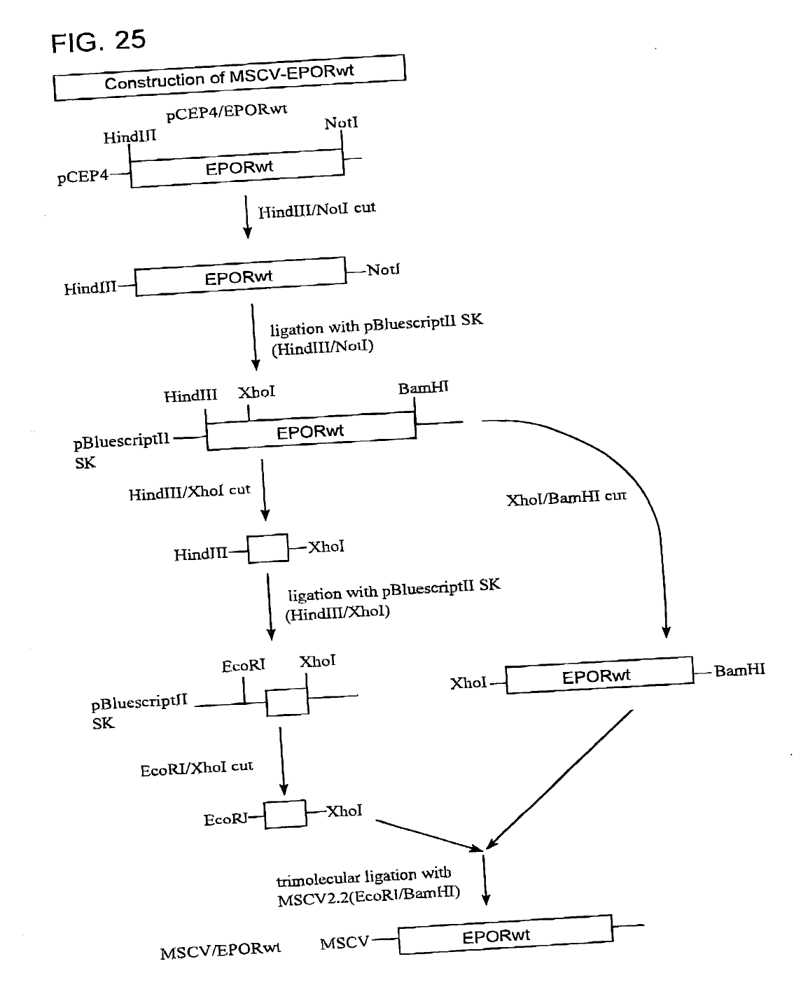

The wild-type human erythropoietin receptor (EPORwt) cDNA was obtained as HindIII-NotI fragment from pCEP4-EPOR (Kralovics R, et al., J. Clin. Invest., 1998; 102(1): 124-129). This fragment was subcloned into HindIII-NotI site of the plasmid pBluescript SK (SK; TOYOBO, Japan) by ligation to construct SK-EPORwt. The HindIII-XhoI fragment encoding a 5′-part of EPOR was subcloned into HindIII-XhoI site of the SKby ligation. The murine phosphoglycerate kinase (pgk) promoter-neomycin phosphotransferase gene (neo) cassette (EcoRI-BamHI) in MSCV2.2 retrovirus (Hawley R G, et al. J. Exp. Med., 1992; 176: 1149-1163) was replaced with the EcoRI-XhoI fragment containing a 5′-part of EPOR and the XhoI-BamHI fragment containing a 3′-part of EPOR to construct MSCV-EPORwt (FIG. 25).

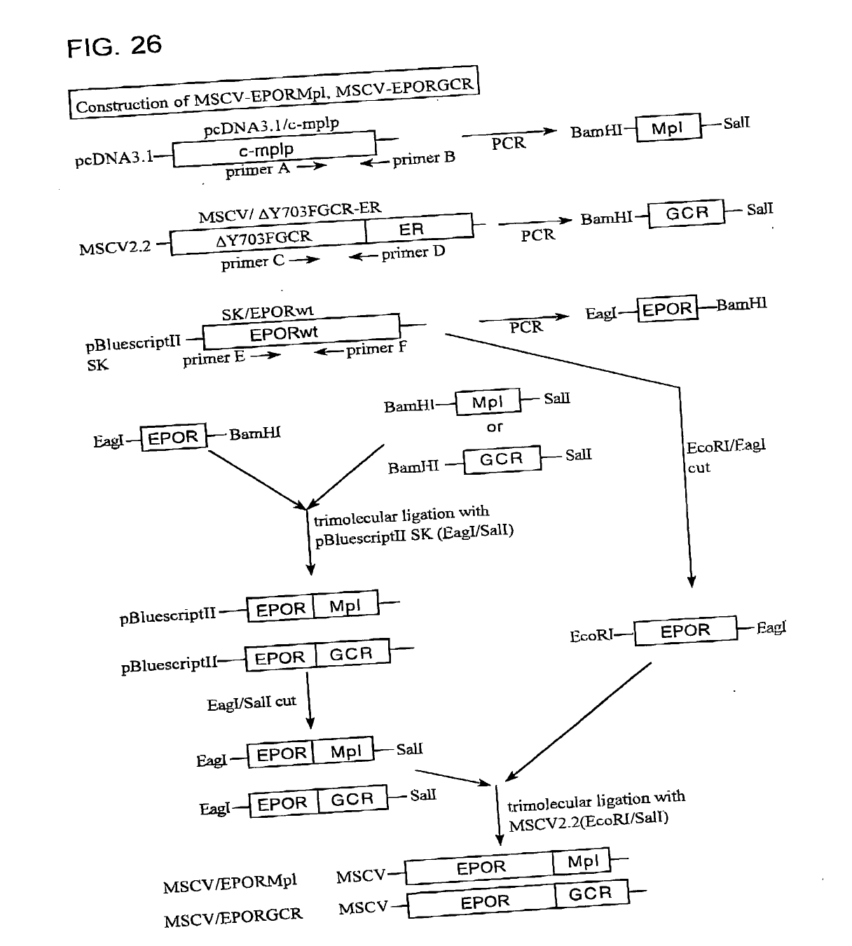

MSCV-EPORMpl and MSCV-EPORGCR were constructed as follow (FIG. 26). The intracellular portion of the human thrombopoietin receptor (Mpl) was derived from pcDNA3.1-c-mpl (The cDNA encoding c-mpl wt was obtained as EcoRV-XbaI fragment from pUC118-c-mpl [TakatokuM, et al. J. Biol. Chem., 1997; 272: 7259-7263]. The c-mpl was cloned into EcoRV-XbaI site of pcDNA3.1 (Invitrogen,), and the resultant vector was designated as pcDNA3.1-c-mpl.), by polymerase chain reaction (PCR) with primer A (5′-AAGGATCCAGGTGGCAGTTTCCTGCA-3′; SEQ ID NO:10) and primer B (5′-CGGTCGACTCAAGGCTGCTGCCAATA-3″; SEQ ID NO:11), creating a BamHI site in the 5′-end and a SalI site in the 3′-end. The intracellular portion of the murine G-CSF receptor (GCR) was derived from MSCV-AY703FGCRER (Matsuda KM, et al., Gene Ther., 1999; 6: 1038-1044), by PCR with primer C (5′-AAGGATCCAAACGCAGAGGAAAGAAGACT-3″; SEQ ID NO:12) and primer D (5′-AAGTCGACCTAGAAACCCCCTTGTTC-3″; SEQ ID NO:13), creating similarly a BamHI site in the 5′-end and a SalI site in the 3′-end. To connect the cytoplasmic portion of EPOR to the intracellular portion of Mpl or GCR, a part of EPOR was constructed by PCR using SK-EPOR with primer E (5′-CTCGGCCGGCAACGGCGCAGGGA-3″; SEQ ID NO:14) and primer F (5′-AAGGATCCCAGCAGCGCGAGCACGGT-3′; SEQ ID NO:15), creating a EagI site in the 5′-end and a BamHI site in the 3′-end. This EPOR fragment (EagI-BamHI) and the BamHI-SalI fragment of Mpl or that of GCR were cloned respectively into EagI-BamHI site of SK by trimolecular ligation. The HindIII-EagI fragment of EPOR from SK-EPORwt was subcloned into HindIII-EagI site of SK-EPORMpl (EagI-SalI) or that of SK-EPORGCR (EagI-SalI) to construct SK-EPORMpl or SK-EPORGCR. Then, pgk and neo cassette (EcoRI-SalI) in MSCV2.2 was replaced with the EcoRI-SalI fragments from SK-EPORMpl or SK-EPORGCR respectively. The resultant constructs were designated as MSCV-EPORMpl and MSCV-EPORGCR.

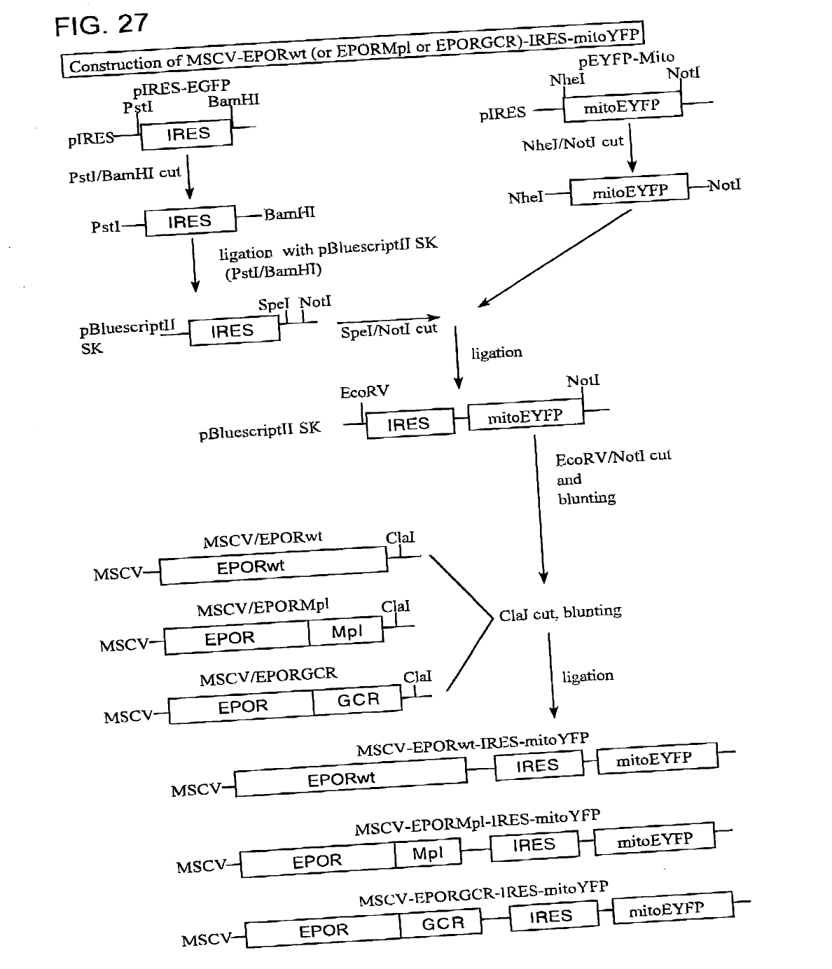

MSCV-EPORwt (or EPORMpl or EPORGCR)-IRES-mitoEYFP was constructed as follows (FIG. 27). SK-ires-mitoEYFP was constructed by inserting IRES (internal ribosome entry site) derived from pIRES-EGFP (Clontech, Palo Alto, Calif.) into PstI-BamHI site of SK and by inserting mitoEYFP derived from pEYFP-Mito (Clontech) into SpeI-NotI site of SK. The EcoRV-NotI blunted fragment encoding IRES-mitoEYFP was cloned into ClaI blunted site of MSCV-EPORwt (or EPOR-Mpl or EPOR-GCR) by ligation, and the resultant constructs was designated as MSCV-EPORwt (or EPORMpl or EPORGCR)-IRES-mitoEYFP. All these constructs were confirmed by sequence analysis.

(2) Cell Lines

Ba/F3 cells (Riken Gene Bank RCB0805, Tsukuba, Japan) were maintained in Dulbecco's modified Eagle medium (DMEM; GIBCO-BRL, Grand Island, N.Y.) supplemented with 10% fetal bovine serum (FBS; GIBCO-BRL), 1% penicillin/streptomycin (GIBCO-BRL), and 10 ng/ml recombinant mouse IL-3 (rmIL-3; GIBCO-BRL). The ecotropic packaging cell line, BOSC23 (AmericanType Culture Collection [ATCC] CRL-11554, Manassas, Va.) andhuman embryonic kidney, 293T cells were maintained in DMEM containing 10% FBS.

(3)Transduction of Ba/F3 Cells

BOSC23 cells were transfected respectively with MSCV-EPORwt-IRES-mitoYFP, MSCV-EPORMpl-IRES-mitoYFP or MSCV-EPORGCR-IRES-mitoYFP using Transfection MBS Mammalian Transfection Kit (Stratagene, La Jolla, Calif.) and the viral supernatants were harvested on day two and three post-transfection. Ba/F3 cells were suspended in 1 ml of viral supernatant containing rmIL-3 at a concentration of 1×10⁵ cells/ml, transferred to the 6-well plates precoated with 20 μg/cm² of human fibronectin fragment (CH296; Takara Shuzo, Otsu, Japan), and then incubated at 37° C. in a humidified atmosphere of 5% CO₂ in air for 48 hours. During this transduction period, viral supernatant was changed four times. As an untransduced control, Ba/F3 cells were similarly treated without using viral supernatant.

(4)Cell Proliferation Assay

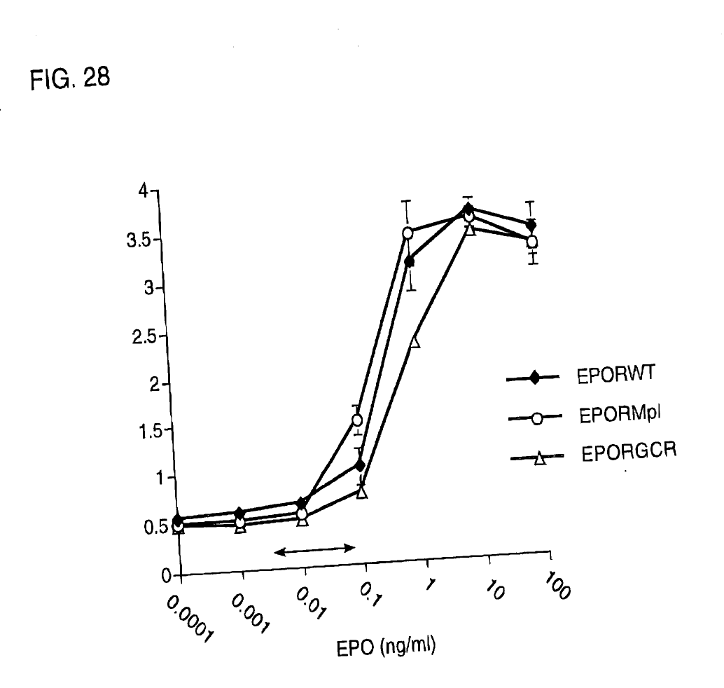

After retroviral infection, transduced Ba/F3 cells were incubated with EPO (long/ml) for five days and almost all cells expressed transgenes respectively. Untransduced or transduced Ba/F3 cells were cultured in the presence or absence of 1 ng/ml rmIL-3 or 10 ng/ml recombinant human erythropoietin (rhEPO). Cell numbers were determined on the days indicated. For EPO dose-response analysis, transduced Ba/F3 cells were stimulated by various concentrations of EPO (FIG. 28). On day zero and day2, cell proliferation assay was performed using CellTier 96 Aqueous One Solution Cell Proliferation Assay {3-(4,5-dimethylthiazol-2-yl)-5-(3-carboxymethoxyphenyl)-2-(4-sulfophenyl)-2H-tetrazolium (MTS); Promega, Madison, Wis.} essentially according to the manufacturer's instruction. In brief, 20 μl per well of MTS-labeling mixture was added. After the incubation at 37° C. for 2 hours, the spectrophotometeical absorbance was measured at the wave length of 490 nm and 650 nm.

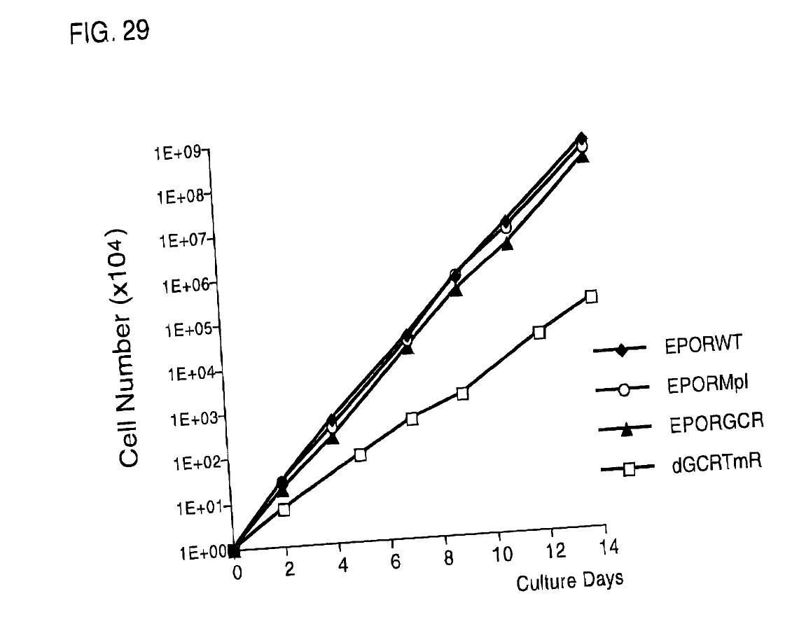

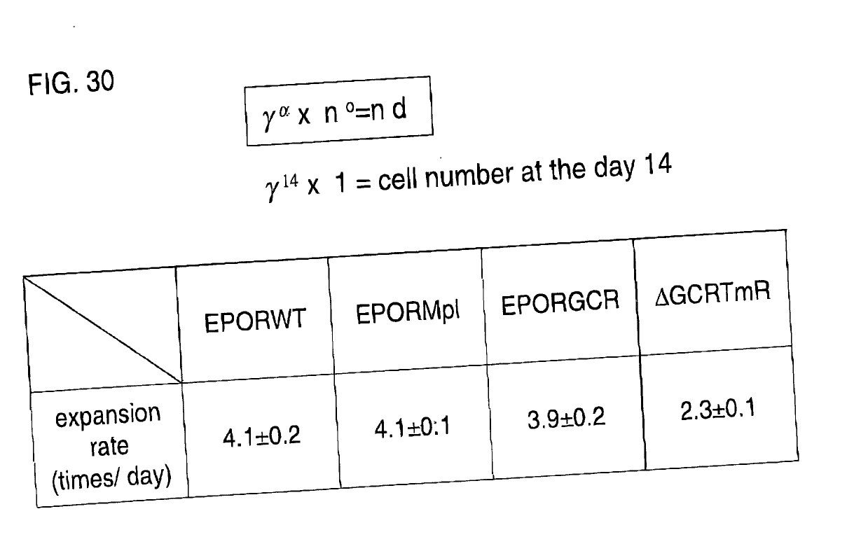

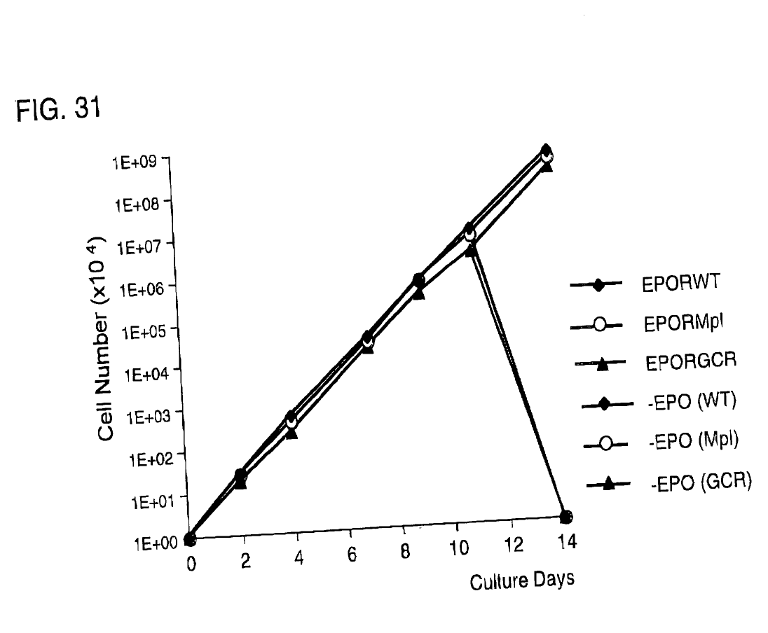

The IL3-dependent Ba/F3 cells expressing the EPO-driven SAGs were cultured in the presence of EPO (long/ml). As control, the Ba/F3 cells expressing the Tm-driven SAG were cultured in the presence of Tm (10-7M). There were no difference among the cells expressing these EPO-driven SAGs, but these cells proliferated at higher level than the cells expressing the Tm-driven SAG (FIGS. 29, 30). Removal of EPOR decreases the number of cells (FIG. 31).

(5)Isolation of Cynomolgus CD34+ Bone Marrow Cells (BMCs)

Clinically healthy adult cynomolgus monkeys born and reared in Tsukuba Primate Center for Medical Science, National Institute of Health, were used for experiments. BMCs were harvested from femora and suspended in lysis buffer to dissolve red blood cells. Immunomagnetic selection of the CD34+ cells was accomplished using Dynabeads system (Dynal AS, Oslo, Norway) according to the manufacturer's instruction.

(6)Transduction of CD34+ Cells

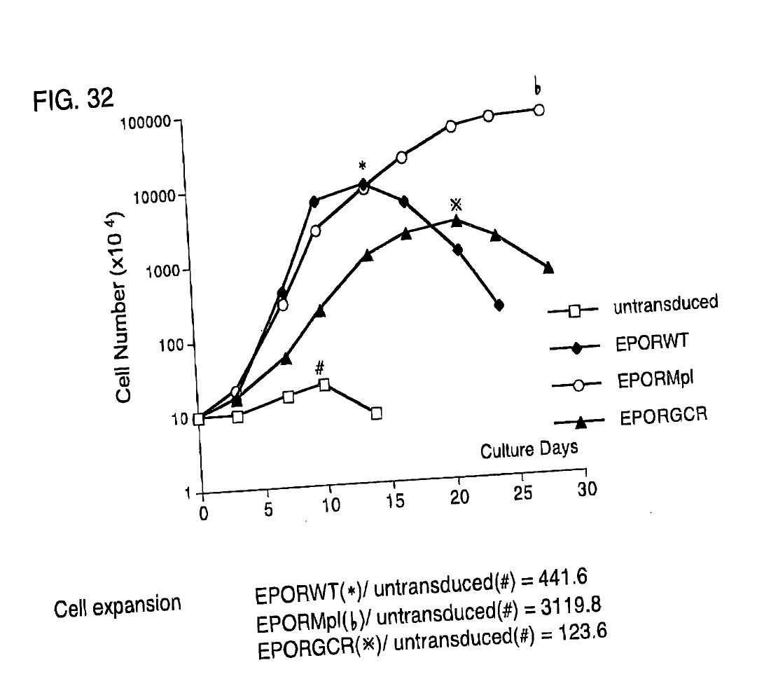

293T cells were co-transfected with MSCV-EPORwt (or EPORMpl or EPORGCR)-IRES-mitoYFP and pCL-Ampho using Transfection MBS Mammalian Transfection Kit (Stratagene, La Jolla, Calif.) and the viral supernatants were harvested on day two and three post-transfection. Cynomolgus CD34+ cells or human CD34+ cells (BioWhittaker) were placed in 6-well plates precoated with 20 μg/cm² of CH296 and were cultured for 24 hours at 37° C. with 5% CO₂ in Iscove's Dulbecco's medium (IMDM) supplemented with 10% FBS, 50 ng/ml recombinant human IL-6 (rhIL6; Ajinomoto, Japan), 100 ng/ml recombinant human SCF (rhSCF; Amgen, Thousand Oaks, Calif.), 100 ng/ml recombinant human Flt-3 ligand (rhFlt-3; Research Diagnostic Inc) and 100 ng/ml recombinant human Thrombopoietin (Kirin, Gumma, Japan). Subsequently, the cells were resuspended in 1 ml of viral supernatant containing all cytokines described above at a concentration of 1×10⁵ cells/ml. During this transduction period, viral supernatant was changed four times. Mock transduction was similarly performed without using a viral supernatant (FIG. 32)

(7) Suspension Culture and Flow Cytometry

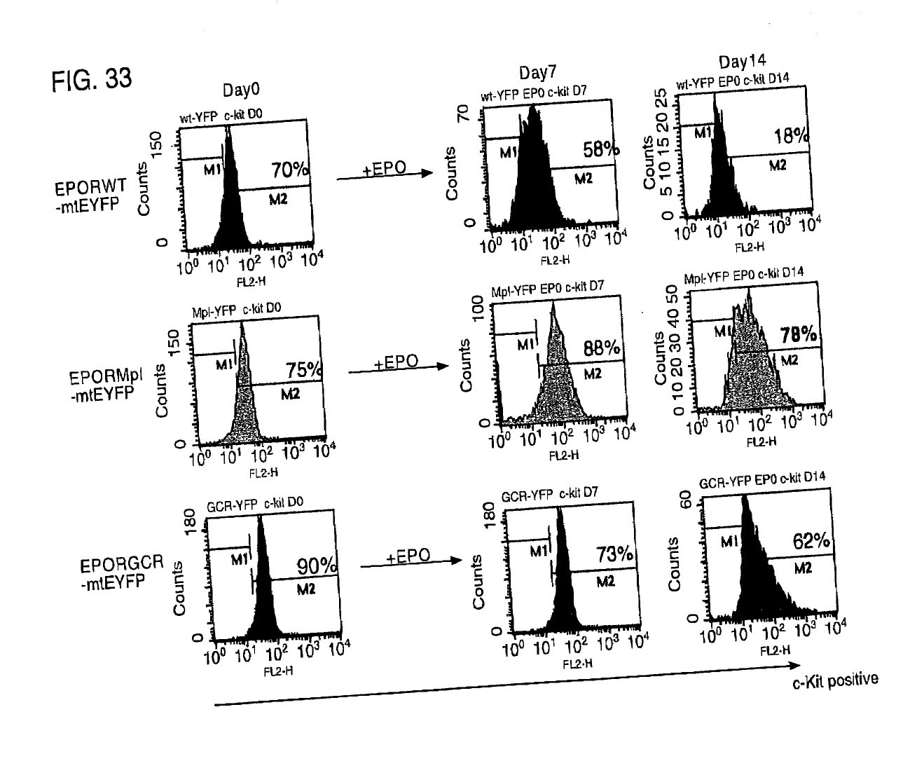

After retroviral transduction, CD34+ cells were washed and aliquot of cells was removed and resuspended in PBS/BSA/Azide, then incubated with directly conjugated primary antibodies phycoerythrin-labeled (PE-labeled) anti-c-kit or PE-labeled anti-CD41 or PE-labeled anti-glycophorin A (Nichirei, Tokyo, Japan) at 4° C. for 20 minutes. The cells were washed once and analyzed using a FACScan flow cytometrer (Becton Dickinson, Palo Alto, Calif.). The rest of cells were cultured in 10% FBS-IMDM containing 100 ng/ml rhFlt-3 ligand or 10 ng/ml recombinant human erythropoietin (rhEpo; Boehringer Mannheim). On every week, aliquots of cells were removed from the suspension cultures and tested by flow cytometry (FIG. 33)

(8) In Vitro Clonogenic Progenitor Assay

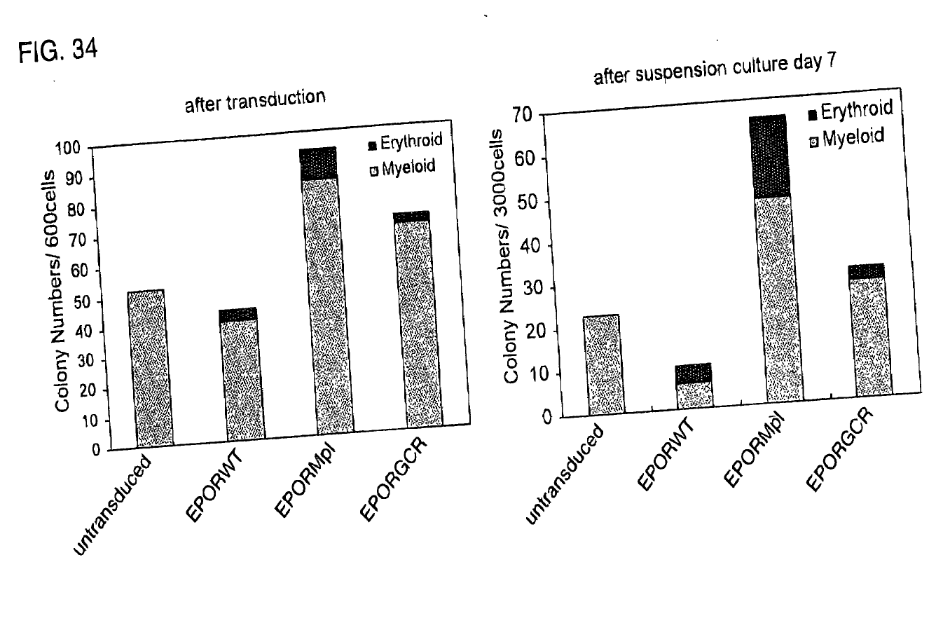

Clonogenic assays were performed in triplicate. The transduced and untransduced cells were plated in 35-mm dish with 1 ml StemPro medium (GIBCO-BRL) in the presence or absence of 20 ng/ml of rhEPO. In some experiments, 100 ng/ml of rmSCF, 100 ng/ml of rhIL-6, 100 ng/ml of rhIL-3 were added to the cultures. After 14 days of incubation at 37° C. in a humidified atmosphere of 5% CO₂ in air, colonies were scored using an inverted microscope and fluorescence microscopy (FIG. 34)

### INDUSTRIAL APPLICABILITY

The present invention has made it possible to selectively amplify a cell into which an exogenous gene has been introduced, in response to an external stimulus, thereby enabling effective gene therapy even when the introduction efficiency of the gene into the target cells is low. Furthermore, since the system for selectively amplifying cells of the present invention can be applied to various blood cells, the range of cells targeted in gene therapy has been widened. Therefore, the present invention provides an important basic technology, particularly in the field of gene therapy.

1. A fusion protein comprising (a) a ligand-binding domain, (b) a domain that associates when a ligand binds to the domain of (a), and (c) a domain comprising a cytokine receptor or a part thereof that imparts proliferation activity to a cell upon the association.

2. The fusion protein of claim 1, wherein the “domain comprising a cytokine receptor or a part thereof that imparts proliferation activity to a cell upon the association” is derived from a G-CSF receptor or c-mpl.

3. The fusion protein of claim 1, wherein the “ligand-binding domain” is derived from a steroid hormone receptor.

4. The fusion protein of claim 3, wherein the steroid hormone receptor is an estrogen receptor.

5. The fusion protein of claim 1, wherein the “ligand” is a tamoxifen, the derivative thereof, or the metabolite thereof and the “ligand-binding domain” and “a domain that associates when a ligand binds to said domain” are derived from a mutant estrogen receptor that is unresponsive to a estrogen and that is responsive to a tamoxifen, the derivative thereof, or the metabolite thereof.

6. A DNA encoding the fusion protein of claim 1.

7. A vector comprising a DNA of claim 6.

8. A cell carrying the vector of claim 7.

9. A method for selectively proliferating the cell of claim 8, which comprises exposing the cell of claim 8 to a ligand capable of acting on the “ligand-binding domain” of the fusion protein of claim 1.

10. A vector comprising a desired exogenous gene and a gene encoding a fusion protein comprising (a) a ligand-binding domain, (b) a domain that associates when a ligand binds to the domain of (a), and (c) a domain that imparts proliferation activity to a cell upon the association.

11. The vector of claim 10, wherein the “domain that imparts proliferation activity to a cell upon the association” is derived from a cytokine receptor.

12. The vector of claim 11, wherein the cytokine receptor is a G-CSF receptor or c-mpl.

13. The vector of claim 10, wherein the “ligand-binding domain” is derived from a steroid hormone receptor.

14. The vector of claim 13, wherein the steroid hormone receptor is an estrogen receptor.

15. The vector of claim 10, wherein the “ligand” is a tamoxifen, the derivative thereof, or the metabolite thereof, and the “ligand-binding domain” and “a domain that associates when a ligand binds to said domain” are derived from a mutant estrogen receptor that is unresponsive to a estrogen and that is responsive to a tamoxifen, the derivative thereof, or the metabolite thereof.

16. The vector of claim 10, wherein the “gene encoding a fusion protein” and the “exogenous gene” are located on the same molecule.

17. The vector of claim 10, wherein the “gene encoding a fusion protein” and the “exogenous gene” are located on separate molecules.

18. A cell carrying the vector of claim 10.

19. A method for selectively proliferating the cell of claim 18, which comprises exposing the cell of claim 18 to a ligand capable of acting on the “ligand-binding domain” of the fusion protein encoded by the gene contained in the vector of claim 10.

20. A kit comprising (a) the vector of claim 7 or claim 10, and (b) a ligand capable of acting on the “ligand-binding domain” of the fusion protein encoded by the gene contained in the vector.


SEQUENCE LISTING (15 sequences)

SEQ ID NO: 1 - DNA, 30 bp, Artificial Sequence
  Artificially Synthesized Primer Sequence
tacgtttaaacgatccgggcacttcaggag

SEQ ID NO: 2 - DNA, 30 bp, Artificial Sequence
  Artificially Synthesized Primer Sequence
ctgtcgacactagtaggagctctcagatcg

SEQ ID NO: 3 - DNA, 24 bp, Artificial Sequence
  Artificially Synthesized Primer Sequence
ctggatccgggcacttcaggagac

SEQ ID NO: 4 - DNA, 26 bp, Artificial Sequence
  Artificially Synthesized Primer Sequence
ctgtcgaccactagtaggagctctca

SEQ ID NO: 5 - DNA, 21 bp, Artificial Sequence
  Artificially Synthesized Primer Sequence
cccacctaccaaggtccctgg

SEQ ID NO: 6 - DNA, 26 bp, Artificial Sequence
  Artificially Synthesized Primer Sequence
cgggatccagaggctgctgccaatag

SEQ ID NO: 7 - DNA, 24 bp, Artificial Sequence
  Artificially Synthesized Primer Sequence
gagtgggtacctgaggcccctagg

SEQ ID NO: 8 - DNA, 26 bp, Artificial Sequence
  Artificially Synthesized Primer Sequence
aactcgaggcagcagagccaggtcac

SEQ ID NO: 9 - DNA, 26 bp, Artificial Sequence
  Artificially Synthesized Primer Sequence
aactcgagaggtggcagtttcctgca

SEQ ID NO: 10 - DNA, 26 bp, Artificial Sequence
  Artificially Synthesized Primer Sequence
aaggatccaggtggcagtttcctgca

SEQ ID NO: 11 - DNA, 26 bp, Artificial Sequence
  Artificially Synthesized Primer Sequence
cggtcgactcaaggctgctgccaata

SEQ ID NO: 12 - DNA, 29 bp, Artificial Sequence
  Artificially Synthesized Primer Sequence
aaggatccaaacgcagaggaaagaagact

SEQ ID NO: 13 - DNA, 26 bp, Artificial Sequence
  Artificially Synthesized Primer Sequence
aagtcgacctagaaacccccttgttc

SEQ ID NO: 14 - DNA, 23 bp, Artificial Sequence
  Artificially Synthesized Primer Sequence
ctcggccggcaacggcgcaggga

SEQ ID NO: 15 - DNA, 26 bp, Artificial Sequence
  Artificially Synthesized Primer Sequence
aaggatcccagcagcgcgagcacggt